(19) United States
(12) Patent Application Publication
(10) Pub. No.: US 2022/0177558 A1
(43) Pub. Date: Jun. 9, 2022

(54) TREATMENT OF TAUPATHY DISORDERS BY TARGETING NEW TAU SPECIES

(71) Applicants: INSERM (INSTITUT NATIONAL DE LA SANTÉ ET DE LA RECHERCHE MÉDICALE), Paris (FR); UNIVERSITÉ DE LILLE, Lille (FR); CENTRE HOSPITALIER REGIONAL UNIVERSITAIRE DE LILLE, Lille (FR)

(72) Inventors: Malika HAMDANE, Lille Cedex (FR); Luc BUEE, Lille Cedex (FR); David BLUM, Lille Cedex (FR); Sabiha EDDARKAOUI, Lille Cedex (FR); Sarah GUEDJDAL, Lille Cedex (FR)

(21) Appl. No.: 17/441,960

(22) PCT Filed: Mar. 24, 2020
(86) PCT No.: PCT/EP2020/058106

§ 371 Date: Sep. 22, 2021

(30) Foreign Application Priority Data
Mar. 25, 2019 (EP) 19305369.1

(51) Int. Cl. C07K 16/18

(52) U.S. Cl. CPC C07K 16/18; C07K 2317/34

(57) ABSTRACT

The invention relates to a method of treatment of Tauopathy disorder using antibody that specifically binds news Tau species, especially Tau species starting from the methionine residue at position (11), said methionine being N-alpha acetylated (AcMet11-Tau). The invention also relates to an antibody that specifically binds this new tau species. Inventors have discovered that AcMet11-Tau is a pathological Tau species that is involved in Tau pathology development. Inventors have demonstrated the causal link between AcMet11-Tau species and Tau pathology by showing that brain expression of AcMet11-Tau species potentiate Tau pathology development in Thy-Tau Transgenic mice and is involved in pathological process, at least by accelerating Tau pathology. Inventors have also used passive immunization approach based on a specific monoclonal antibody (2H2D11) in order to demonstrate that the reduction/neutralization of this Tau species in the Thy-Tau22 transgenic model of Tau pathology lead to protective effect towards Tau pathology and associated memory deficits. Furthermore, the inventors subcloned 2C12 hybridoma and selected the 2C1C8, a further antibody against N-alpha-acetyl-Met11-Tau (AcMet11-Tau). They demonstrate that the 2C1C8 antibody displayed also specificity towards N-alpha-terminally acetylated methionine11 of Tau protein and labels neurons displaying neurofibrillary degeneration in the hippocampus of Thy-Tau22 transgenic mice.

### FIELD OF THE INVENTION

The invention relates to a method of treatment of tauopathy disorder using antibody that specifically binds news Tau species, especially Tau species starting from the methionine residue at position 11, said methionine being N-alpha acetylated (AcMet11-Tau). The invention further relates to the specific monoclonal antibody 2H2D11 and derivatives. These specific antibodies can be used for the therapy of Tauopathy disorders such as Alzheimer disease.

### BACKGROUND OF THE INVENTION

Tau proteins belong to the microtubule-associated proteins family and are found essentially in neurons where they are mainly involved in regulation of microtubules stability and dynamics as well as axonal transport. There are six Tau isoforms in human adult brain with different N-termini and containing in their C-terminal part either 4 microtubule-binding domains (4R isoforms) or 3 microtubule-binding domains (3R isoforms). Tau proteins derive from a single gene by alternative splicing of exons 2, 3 and 10 (Caillet-Boudin et al., 2015). Tau proteins aggregate into filaments in a large group of neurodegenerative disorders referred to as Tauopathies. Alzheimer's disease (AD) is the most common Tauopathy and form of dementia. One of its neuropathological hallmarks is the neurofibrillary degeneration (NFD) that is made of aggregated Tau proteins bearing abnormal post-translational modifications. The progression of NFD in cortical brain area is closely correlated to cognitive impairment in AD (Braak and Braak, 1995; Duyckaerts et al, 1998; Delacourte et al., 1999), indicating hence that Tau is a central player in AD pathology and valuable therapeutic target (Novak et al., 2018). Immunotherapy is currently an innovative and a promising therapeutic approach to consider for encountering AD (Wisniewski et al., 2014). Passive immunotherapies in rodent models of Tauopathies have shown that monoclonal antibodies injected via the systemic pathway can cross the blood-brain barrier and bind to Tau-targeted proteins (Pedersen et al., 2015; Ittner et al., 2015). The mechanisms underlying the beneficial effect of Tau-based immunotherapy in mice models are still to be determined. The therapeutic antibody could either block Tau seeding avoiding hence formation of pathologic oligomers and aggregated Tau species, or block intercellular Tau spreading, or facilitate Tau degradation via endosomal/lysosomal pathway (Sigurdsson et al., 2016). However, the challenge for Tau immunotherapy relies on the identification of Tau species to be targeted without affecting the normal Tau proteins and thus avoid the detrimental side effects.

Finally, there is a continued need to develop novel immunotherapy for tau disorders.

Among Tau species found in AD brains, truncated forms are likely to be toxic ones but their role in pathological process have been under-investigated (Zilka et al., 2012). Inventors have recently identified new N-terminally truncated Tau species from the human brain (Derisbourg et al., 2015). Among these new species, Tau protein starting at residue Met11 (Met11-Tau) is of particular interest since Met11 is located in the region encoded by exon1 that is shared by all Tau protein isoforms. Even though little is known about exon1 functions, its modifications could impact on Tau functions and result in Tau pathology (Hayashi et al., 2002; Poorkaj et al., 2002; Magnani et al., 2007; Derisbourg et al., 2015). The role of N-terminus of Tau would be at least related to its involvement in the formation of a particular Tau structure. Indeed, Tau that is a natively unfolded protein likely adopts a “paperclip” conformation as a result of intra-molecular interaction between N-terminal and C-terminal domains (Carmel et al., 1996; Jeganathan et al., 2006; Jeganathan et al., 2008). Therefore, loss of the outermost N-terminus of Tau, as encountered in Met11-Tau protein, would be of crucial functional and pathological consequences. More interestingly, our further analyses of proteomics data have showed that Met11-Tau is also detected in an N-alpha-terminally acetylated form (AcMet11-Tau). Importantly, these Tau species have never been described before. Inventors have first developed a monoclonal antibody allowing for the specific detection of AcMet11-Tau species. Then, the antibody was used to establish an association between AcMet11-Tau species and Tau pathology using the Thy-Tau22 transgenic mouse model that progressively develops neurofibrillary degeneration (NFD) and memory deficits (Schindowski et al., 2006; Van der Jeugd at al., 2013). AcMet11-Tau species were clearly detected in neurons displaying NFD on hippocampal brain sections of Thy-Tau22 mice. Interestingly, AcMet11-Tau epitope was detected at early stages of the pathological process that precede memory deficits. Furthermore, immunohistochemistry analysis of human brain hippocampus sections showed that the antibody labels neurofibrillary tangles in AD brains while it is not reactive in hippocampus from elderly controls (WO 2018/178078). Overall, our data indicate that AcMet11-Tau epitope is clearly detected in neurons displaying NFD and represent new and early pathological Tau species of physiopathological and therapeutic value.

### SUMMARY OF THE INVENTION

The invention provide an anti Tau antibodies wherein said antibody binds to an epitope comprising the following amino acid sequences (N-α-acetyl)MEDHAGTYGLG (SEQ ID NO:8) for use in therapy.

In a particular embodiment anti Tau antibody of the invention is used in the treatment of tauopathy.

The invention further relates to a specific anti AcMet11-Tau antibody and derivatives.

### DETAILED DESCRIPTION OF THE INVENTION

Previous data of the inventors have shown AcMet11-Tau to be a biomarkers signature feature of AD related-Tau pathology (see WO 2018/178078).

Inventors have now performed further analyses and data indicate that AcMet11-Tau are pathological Tau species that are involved in Tau pathology development.

First, inventors have done biochemical fractionation of AD brain proteins. ELISA analyses showed that like pathological hyperphosphorylated Tau proteins, AcMet11-Tau species are present in the insoluble fraction (which contains Tau protein aggregates) (FIG. 1)

Second, in THY-Tau22 mice that gradually develop hippocampal Tau pathology from 3 to 10 months and memory deficits from 6 months (Schindowski et al., 2006; Van der Jeugd at al., 2013), AcMet11-Tau species are detected at early stages of the pathological process that precede memory deficits. (FIG. 2).

Third, inventors have demonstrated the causal link between AcMet11-Tau species and Tau pathology by showing that brain expression of AcMet11-Tau species potentiate Tau pathology development in Thy-Tau Transgenic mice (FIGS. 3 and 4), and are involved in pathological process, at least by accelerating Tau pathology.

Fourth, inventors have used passive immunization approach based on the specific monoclonal antibody previously developed (2H2D11 IgG2a isotype) in order to demonstrate that the reduction/neutralization of these Tau species in the Thy-Tau22 transgenic model of Tau pathology lead to protective effect towards Tau pathology (FIGS. 7 and 8) and associated memory deficits (FIG. 10)

Overall, these data indicate that AcMet11-Tau are early pathological species of physiopathological value and could hence be a valuable therapeutic target.

Antibodies that Specifically Binds News Tau Species for Use in Therapy

The invention relates to an anti Tau antibody, wherein said antibody binds to an epitope comprising the following amino acid sequences (N-α-acetyl)MEDHAGTYGLG (SEQ ID NO:8) for use in therapy.

In particular embodiment the anti Tau antibody for use according to the invention specifically binds Tau polypeptide starting from the methionine residue at position 11 wherein said methionine at position 11 is N-alpha acetylated (AcMet11-Tau).

In particular embodiment, the anti Tau antibody for use according to the invention specifically binds a polypeptide selected from the group comprising or consisting of:
(i) the amino acids sequence consisting of Tau N-Alpha Acetyl-Met11-352 (SEQ ID NO:1);
(ii) the amino acids sequence consisting of Tau N-Alpha Acetyl-Met11-381 (SEQ ID NO:2);
(iii) the amino acids sequence consisting of Tau N-Alpha Acetyl-Met11-383 (SEQ ID NO:3)
(iv) the amino acids sequence consisting of Tau N-Alpha Acetyl-Met11-410 (SEQ ID NO:4)
(v) the amino acids sequence consisting of Tau N-Alpha Acetyl-Met11-412 (SEQ ID NO:5);
(vi) the amino acids sequence consisting of Tau N-Alpha Acetyl-Met11-441 (SEQ ID NO:6);
(vii) the amino acids sequence consisting of Tau N-Alpha Acetyl-Met11-776 (SEQ ID NO:7);
(viii) a fragment of at least 9 consecutive amino acids starting from the N-Alpha Acetyl methionine residue at position 11 of the sequence of (i), to (vii)).

In particular embodiment, the anti Tau antibody for use according to the invention specifically binds a polypeptide selected from the group comprising or consisting of:
(i) the amino acids sequence consisting of Tau N-Alpha Acetyl-Met11-352 (SEQ ID NO:1);
(ii) the amino acids sequence consisting of Tau N-Alpha Acetyl-Met11-381 (SEQ ID NO:2);
(iii) the amino acids sequence consisting of Tau N-Alpha Acetyl-Met11-383 (SEQ ID NO:3)
(iv) the amino acids sequence consisting of Tau N-Alpha Acetyl-Met11-410 (SEQ ID NO:4)
(v) the amino acids sequence consisting of Tau N-Alpha Acetyl-Met11-412 (SEQ ID NO:5);
(vi) the amino acids sequence consisting of Tau N-Alpha Acetyl-Met11-441 (SEQ ID NO:6);
(vii) the amino acids sequence consisting of Tau N-Alpha Acetyl-Met11-776 (SEQ ID NO:7);
(viii) a fragment of at least 11 consecutive amino acids starting from the N-Alpha Acetyl methionine residue at position 11 of the sequence of (i), to (vii)).

The term “Tau” as used herein denotes the Tau protein from mammals and especially from primates (and Tupaiidae). Human Tau is a neuronal microtubule-associated protein found predominantly in axons and functions to promote tubulin polymerization and stabilize microtubules. Six isoforms (isoform A, B, C, D, E, F, G, fetal-Tau) are found in the human brain, the longest isoform comprising 441 amino acids (isoform F, Uniprot P10636-8). Tau and its properties are also described by Reynolds, C. H. et al., J. Neurochem. 69 (1997) 191-198. Tau, in its hyperphosphorylated form, is the major component of paired helical filaments (PHF), the building block of neurofibrillary lesions in Alzheimer's disease (AD) brain. Tau can be phosphorylated at its serine or threonine residues by several different kinases including GSK3beta, cdk5, MARK and members of the MAP kinase family.

The protein sequence of human Tau protein, and its isoforms, may be found in Uniprot database with the following access numbers:

Tau isoform Fetal (352 Amino Acids) Uniprot P10636-2

Tau isoform B (381 AA) Uniprot P10636-4

Tau isoform D (383 AA) Uniprot P10636-6

Tau isoform C (410 AA) Uniprot P10636-5

Tau isoform E (412 AA) Uniprot P10636-7

Tau isoform F (441 AA) Uniprot P10636-8

Tau isoform G (776 AA) Uniprot P10636-9

Truncation is an additional post-translational modification that could have an etiological role in Tau pathology. Numerous carboxy-truncated forms of Tau protein have been also described that could impacts biochemical and functional properties of Tau protein and triggers a gain of toxic function (Garcia-Sierra et al., 2001; Rissman et al., 2004; Zilka et al., 2006; Basurto-Islas et al., 2008; McMillan et al., 2011).

In some embodiments, the tau polypeptide specifically detected by the antibodies of the invention comprises at most 766 amino acids (and at least 9). In some embodiments, the polypeptide of the invention comprises 766, 765, 764, 763, 762, 761, 760, 759, 758, 757, 756, 755, 754, 753, 752, 751, 750, 749, 748, 747, 746, 745, 744, 743, 742, 741, 740, 739, 738, 737, 736, 735, 734, 733, 732, 731, 730, 729, 728, 727, 726, 725, 724, 723, 722, 721, 720, 719, 718, 717, 716, 715, 714, 713, 712, 711, 710, 709, 708, 707, 706, 705, 704, 703, 702, 701, 700, 699, 698, 697, 696, 695, 694, 693, 692, 691, 690, 689, 688, 687, 686, 685, 684, 683, 682, 681, 680, 679, 678, 677, 676, 675, 674, 673, 672, 671, 670, 669, 668, 667, 666, 665, 664, 663, 662, 661, 660, 659, 658, 657, 656, 655, 654, 653, 652, 651, 650, 649, 648, 647, 646, 645, 644, 643, 642, 641, 640, 639, 638, 637, 636, 635, 634, 633, 632, 631, 630, 629, 628, 627, 626, 625, 624, 623, 622, 621, 620, 619, 618, 617, 616, 615, 614, 613, 612, 611, 610, 609, 608, 607, 606, 605, 604, 603, 602, 601, 600, 599, 598, 597, 596, 595, 594, 593, 592, 591, 590, 589, 588, 587, 586, 585, 584, 583, 582, 581, 580, 579, 578, 577, 576, 575, 574, 573, 572, 571, 570, 569, 568, 567, 566, 565, 564, 563, 562, 561, 560, 559 558, 557, 556, 555, 554, 553, 552, 551, 550, 549, 548, 547, 546, 545, 544, 543, 542, 541, 540, 539, 538, 537, 536, 535, 534, 533, 532, 531, 530, 529, 528, 527, 526, 525, 524, 523, 522, 521, 520, 519, 518, 517, 516, 515, 514, 513, 512, 511, 510, 509, 508, 507, 506, 505, 504, 503, 502, 501, 500, 499, 498, 497, 496, 495, 494, 493, 492, 491, 490, 489, 488, 487, 486, 485, 484, 483, 482, 481, 480, 479, 478, 477, 476, 475, 474, 473, 472, 471, 470, 469, 468, 467, 466, 465, 464, 463, 462, 461, 460, 459, 458, 457, 456, 455, 454, 453, 452, 451, 450, 449, 448, 447, 446, 445, 444, 443; 442, 441, 440, 439, 438, 437, 436, 435, 434, 433, 432, 431, 430, 429, 428, 427, 426, 425, 424, 423, 422, 421, 420, 419, 418, 417, 416, 415, 414, 413, 412, 411, 410, 409, 408, 407, 406, 405, 404, 403, 402, 401, 400, 399, 398, 397, 396, 395, 394, 393, 392, 391, 390, 389, 388, 387, 386, 385, 384, 383, 382, 381, 380, 379, 378, 377, 376, 375, 374, 373, 372, 371, 370, 369, 368, 367, 366, 365, 364, 363, 362, 361, 360, 359, 358, 357, 356, 355, 354, 353, 352, 351, 350, 349, 348, 347, 346, 345, 344, 343, 342, 341, 340, 339, 338, 337, 336, 335, 334, 333, 332, 331, 330, 329, 328, 327, 326, 325, 324, 323, 322, 321, 320, 319, 318, 317, 316, 315, 314, 313, 312, 311, 310, 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273 272, 271, 270, 269, 268, 267, 266, 265, 264 263, 262, 261, 260, 259, 258, 257, 256, 255, 254, 253, 252, 251, 250, 249, 248, 247, 246, 245, 244, 243, 242, 241, 240, 239, 238, 237, 236, 235, 234, 233, 232, 231, 230, 229, 228, 227, 226, 225, 224, 223, 222, 221, 220, 219, 218, 217, 216, 215, 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100; 99; 98; 97; 96; 95; 94; 93; 92; 91; 90; 89; 88; 87; 86; 85; 84; 83; 82; 81; 80; 79; 78; 77; 76; 75; 74; 73; 72; 71; 70; 69; 68; 67; 66; 65; 64; 63; 62; 61; 60; 59; 58; 57; 56; 55; 54; 53; 52; 51; 50; 49; 48; 47; 46; 45; 44; 43; 42; 41; 40; 39; 38; 37; 36; 35; 34; 33; 32; 31; 30; 29; 28; 27; 26; 25; 24; 23; 22; 21; 20; 19; 18; 17; 16; 15; 14; 13; 12; 11; 10 or 9 amino acids. In some embodiments, the polypeptide of the invention comprises less than 50 amino acids. In some embodiments, the polypeptide of the invention comprises less than 30 amino acids. In some embodiments, the polypeptide of the invention comprises less than 25 amino acids. In some embodiments, the polypeptide of the invention comprises less than 20 amino acids. In some embodiments, the polypeptide of the invention comprises less than 15 amino acids.

The inventors have generated specific antibodies directed against the Tau polypeptide AcMet11-Tau.

The monoclonal antibodies were produced by immunizing mice with the synthetic peptides, (N-α-acetyl)MEDHAGTYGLG (SEQ ID NO:8). More precisely, the inventors have found that antibodies screened for their capacity to bind specifically AcMet11-Tau polypeptides (Tau polypeptides starting from the methionine residue at position 11 wherein said methionine at position 11 is N-alpha acetylated) and to stain cell lines samples as well as brain samples from AD patients and from THY-Tau22 mouse model of tauopathies (FIGS. 1 and 2). Screening step of the antibodies of the invention has shown that these antibodies are specific of N-alpha acetylated form of Methionine 11 Tau species because they did not bind to the non-acetylated form of Methionine 11 Tau species nor to non-truncated Tau species, neither to the N-alpha-acetyl-Met1-Tau polypeptides (non amino-truncated Tau species)

The invention provides an antibody that specifically binds to a Tau polypeptide AcMet11-Tau especially to the epitope located within the peptide (N-α-acetyl)MEDHAGTYGLG (SEQ ID NO:8). Such antibodies are characterized in that they specifically bind to N-alpha-acetyl-Met11-Tau species.

In a particular embodiment the antibody of the invention does not bind to a non N-alpha-acetylated form of Methionine 11 Tau polypeptide (i.e SEQ ID No 9) and/or a N-alpha-acetyl-Met1-Tau polypeptide (i.e SEQ ID No 10).

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated VL-CDR1, VL-CDR2, VL-CDR3 and VH-CDR1, VH-CDR2, VH-CDR3, respectively. An antigen-binding site, therefore, includes six CDRs, comprising the CDR set from each of a heavy and a light chain V region. Framework Regions (FRs) refer to amino acid sequences interposed between CDRs.

Antibody binding to AcMet11-Tau polypeptide can be assayed by conventional methods known in the art. The mature form of polypeptide of the invention is preferably used for assaying antibody binding to epitope of polypeptide of the invention (especially to Ac-Met11 epitope: (N-α-acetyl)MEDHAGTYGLG (SEQ ID NO:8). Alternatively, any variant form of polypeptide of the invention that retains binding of mAb 2H2D11 can be used. Many different competitive binding assay format(s) can be used for determining epitope binding. The immunoassays which can be used include, but are not limited to, competitive assay systems using techniques such as radioimmunoassays, ELISA, “sandwich” immunoassays, immunoprecipitation assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York). For example, the BIACORE® (GE Healthcare, Piscataway, N.J.) is one of a variety of surface plasmon resonance assay formats that are routinely used to epitope bin panels of monoclonal antibodies. Additionally, routine cross-blocking assays such as those described in Antibodies, A Laboratory Manual, Cold Spring Harbor Laboratory, Ed Harlow and David Lane, 1988, can be performed. An example of a suitable ELISA assay is also described in the Example below.

As used herein, the term “Affinity” refers to the strength of interaction between antibody and antigen (especially to Ac-Met11 antigen: (N-α-acetyl)MEDHAGTYGLG (SEQ ID NO:8) at single antigenic sites. Within each antigenic site, the variable region of the antibody “arm” interacts through weak non-covalent forces with the antigen at numerous sites; the more interactions, the stronger the affinity. Affinity can be determined by measuring K_{D}. The term “K_{D}”, as used herein, is intended to refer to the dissociation constant, which is obtained from the ratio of K_{d} to Kₐ(i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

These antibodies can be polyclonal or monoclonal. When the antibodies are monoclonal, they can for example correspond to chimeric, humanized or fully human antibodies, antibody fragment and single domain antibody.

The term “chimeric antibody” refers to an antibody which comprises a VH domain and a VL domain of an antibody, and a CH domain and a CL domain of a human antibody.

According to the invention, the term “humanized antibody” refers to an antibody having variable region framework and constant regions from a human antibody but retains the CDRs of a previous non human antibody.

The term “antibody fragment” refers to a fragment of an antibody which contain the variable domains comprising the CDRs of said antibody. The basic antibody fragments include Fab, Fab′, F(ab′)2 Fv, scFv, dsFv. For example of antibody fragment see also for review, Holliger et al Nature Biotechnology 23, issue 9 1126-1136 (2005), which are includes herein by reference.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

As used herein the term “single domain antibody” has its general meaning in the art and refers to the single heavy chain variable domain of antibodies of the type that can be found in Camelid mammals which are naturally devoid of light chains. Such single domain antibody is also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. VHHs have a molecular weight of about one-tenth of human IgG molecule ones and have a physical diameter of only a few nanometers. One consequence of the small size is the ability of single domain antibodies (or VHHs) to bind to antigenic sites that are functionally invisible to larger antibody proteins, i.e., single domain antibody (or VHHs) are useful as reagents to detect antigens that are otherwise cryptic using classical immunological techniques, and as possible therapeutic agents. Thus yet another consequence of small size is that a single domain antibody (or VHH) can inhibit activity/interactions as a result of binding to a specific site in a groove or narrow cleft of a target protein, and hence can serve in a capacity that more closely resembles the function of a classical low molecular weight drug than that of a classical antibody. The low molecular weight and compactness of the fold result in VHHs being extremely thermostable, stable to extreme pH and to proteolytic digestion, and the absence of Fc fragment provides a low antigenic character. Another consequence is that VHHs readily move from the circulatory system into tissues, and have a higher probability to cross the blood-brain barrier and can treat disorders that affect nervous tissue. Single domain antibodies (or VHHs) can further facilitate drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH (variable heavy chain) domains given by the International ImMunoGeneTics information system aminoacid numbering (http://imgt.org/).

Methods for obtaining such antibodies are well known in the art. For example, monoclonal antibodies according to the invention can be obtained through immunization of a non-human mammal with said fragment comprising or consisting of any one of (i) to (viii). Starting from the polyclonal antibodies, one can then obtain monoclonal antibodies using standard methods.

A further object of the invention relates to an antibody of the invention for use in the treatment of tauopathies.

The term “tauopathy” has its general meaning in the art and refers to a disease characterized by Tau aggregation (Iqbal, K. et al. Biochimica et Biophysica Acta (BBA) 1739 (2005) 198-210). Tauopathies include among others, Alzheimer's Disease, Down syndrome; Guam parkinsonism dementia complex; Dementia pugilistica and other chronic traumatic encephalopathies; myotonic dystrophies; Niemann-Pick disease type C; Pick disease; argyrophilic grain disease; Fronto-temporal dementia; Cortico-basal degeneration; Pallido-ponto-nigral degeneration; Progressive supranuclear palsy; and Prion disorders such as Gerstmann-Sträussler-Scheinker disease with tangles.

In a particular embodiment, the Tauopathy disorders is Alzheimer's Disease.

Specific Antibody of the Invention

The inventors have cloned and sequenced the variable domain (VL) of the light chain, and the variable domain (VH) of the heavy chain of the monoclonal antibody 2H2D11. The location of the sequences encoding the complementarity determining regions (CDRs) of said antibody have been determined according to the IMGT numbering system. The IMGT unique numbering has been defined to compare the variable domains whatever the antigen receptor, the chain type, or the species (Lefranc M.-P., Immunology Today, 18, 509 (1997); Lefranc M.-P., The Immunologist, 7, 132-136 (1999); Lefranc, Dev. Comp. Immunol., 27, 55-77 (2003)).

In one embodiment, the invention relates to an anti Tau antibody comprises:

(a) a heavy chain wherein the variable domain comprises:
a H-CDR1 having 90, 91, 92, 93, 94, 95, 96, 97, 98 or 99% of identity with the sequence set forth as SEQ ID NO: 11, and
a H-CDR2 having 90, 91, 92, 93, 94, 95, 96, 97, 98 or 99% of identity with the sequence set forth as SEQ ID NO: 12, and
a H-CDR3 having 90, 91, 92, 93, 94, 95, 96, 97, 98 or 99% of identity with the sequence set forth as SEQ ID NO: 13;

(b) a light chain wherein the variable domain comprises:
a L-CDR1 having 90, 91, 92, 93, 94, 95, 96, 97, 98 or 99% of identity with the sequence set forth as SEQ ID NO: 14, and
a L-CDR2 having 90, 91, 92, 93, 94, 95, 96, 97, 98 or 99% of identity with the sequence set forth as SEQ ID NO: 15, and
a L-CDR3 having 90, 91, 92, 93, 94, 95, 96, 97, 98 or 99% of identity with the sequence set forth as SEQ ID NO: 16.

(c) that binds to AcMet11-Tau polypeptides with substantially the same affinity as an antibody having a variable light chain domain (VL) and/or a variable heavy chain domain (VH) of the antibody 2H2D11.

In one embodiment, the antibody of the present invention comprises:

(a) a heavy chain wherein the variable domain comprises:
a H-CDR1 having a sequence set forth as SEQ ID NO: 11, and
a H-CDR2 having a sequence set forth as SEQ ID NO: 12, and
a H-CDR3 having a sequence set forth as SEQ ID NO: 13;

(b) a light chain wherein the variable domain comprises:
a L-CDR1 having a sequence set forth as SEQ ID NO: 14, and
a L-CDR2 having a sequence set forth as SEQ ID NO: 15, and
a L-CDR3 having a sequence set forth as SEQ ID NO: 16.

In one embodiment, the antibody of the present invention comprises:
a heavy chain wherein the variable domain has at least 70% of identity with the sequence set forth as SEQ ID NO:17
a light chain wherein the variable domain has at least 70% of identity with the sequence set forth as SEQ ID NO:18

and that binds to AcMet11-Tau polypeptide with substantially the same affinity as an antibody having a variable light chain domain (VL) and/or a variable heavy chain domain (VH) of the antibody 2H2D11.

In one embodiment, the antibody of the present invention comprises:
a heavy chain wherein the variable domain has at least 80% of identity with the sequence set forth as SEQ ID NO:17
a light chain wherein the variable domain has at least 80% of identity with the sequence set forth as SEQ ID NO:18

and binds to AcMet11-Tau with substantially the same affinity as an antibody having a variable light chain domain (VL) and/or a variable heavy chain domain (VH) of the antibody 2H2D11.

In one embodiment, the antibody of the present invention comprises:
a heavy chain wherein the variable domain has 90, 91, 92, 93, 94, 95, 96, 97, 98 or 99% of identity with the sequence set forth as SEQ ID NO:17
a light chain wherein the variable domain has 90, 91, 92, 93, 94, 95, 96, 97, 98 or 99% of identity with the sequence set forth as SEQ ID NO:18

and binds to AcMet11-Tau polypeptide with substantially the same affinity as an antibody having a variable light chain domain (VL) and/or a variable heavy chain domain (VH) of the antibody 2H2D11.

In one embodiment, the antibody of the present invention comprises:
a heavy chain wherein the variable domain has a sequence set forth as SEQ ID NO:17 and
a light chain wherein the variable domain has a sequence set forth as SEQ ID NO:18

In some embodiments the antibody (2H2D11 derivative) according to the invention comprises a H-CDR1 having a sequence set forth as SEQ ID NO: 11, a H-CDR2 having a sequence set forth as SEQ ID NO:12 and a H-CDR3 having a sequence set forth as SEQ ID NO:13, a L-CDR1 having a sequence set forth as SEQ ID NO: 14, a L-CDR2 having a sequence set forth as SEQ ID NO:15 and a L-CDR3 having a sequence set forth as SEQ ID NO:16.

In a particular embodiment, the antibodies described above bind to the same antigen and have the same or improved properties (see definition of “2H2D11 analogue”) of the antibody of the invention i.e. the antibody with the CDRs of SEQ ID NO: 11 to 16.

In a particular embodiment, the antibody of the present invention (such as 2H2D11 or Analogue or Derivative and antibody for use in therapy that bind to Ac-Met11 epitope: (N-α-acetyl)MEDHAGTYGLG (SEQ ID NO:8)) is able to inhibit pathological seeding and/or aggregation of Tau protein through experiments.

As used herein, a “2H2D11 analogue” or “2H2D11 derivative” refers to an antibody exhibiting at least the same, or better, binding to Tau protein (especially to Ac-Met11 epitope: (N-α-acetyl)MEDHAGTYGLG (SEQ ID NO:8)) and at least one of the biological activities of an antibody 2H2D11 with a VH of SEQ ID NO: 17 an VL of SEQ ID NO: 18.

The antibody of the present invention may for example be characterized in that it is capable of inhibiting pathological seeding and/or aggregation of Tau protein through experiments (see: Aggregation seeding assays in HEK293 reporter cell-line). Briefly such assay is based on the sensor cell line constitutively expressing Tau RD (MTBD), with a P301 S mutation, fused to either CFP (Cyan Fluorescent Protein) or YFP (Yellow Fluorescent Protein) that together generate a FRET (Forster Resonance Energy Transfer) signal upon induction of MTBD-P301 S aggregation. The intracellular aggregation of MTBD-P301 S protein is induced in the presence of Tau seeds, leading to a FRET signal (Holmes et al. 2014).

The biological activities of the antibody of the invention are, for example, to reduce the level of pathological seeding and/or aggregation of Tau protein as described above. The evaluation of the Tau pathological seeding and/or aggregation level allows to determine the therapeutic properties of the antibody such as the correction of cognitive impairment observed in tauopathies.

Sequences for the variable heavy chain (VH) and variable light chain (VH) and domains (CDRs) of antibody 2H2D11 are indicated in the following Table 1:

| Mab 2H2D11 | Sequence |
|---|---|
| H-CDR1 | DYYLS |
| (SEQ ID NO: 11) | |
| H-CDR2 | NRADGYTTNYSASVKG |
| (SEQ ID NO: 12) | |
| H-CDR3 | DNDHYAMDY |
| (SEQ ID NO: 13) | |
| L-CDR1 | KSSQSLLYSNGKTYLN |
| (SEQ ID NO: 14) | |
| L-CDR2 | QVSKLDP |
| (SEQ ID NO: 15) | |
| L-CDR3 | LQGTYYPYT |
| (SEQ ID NO: 16) | |
| VH | EVRLVESGGGLVQPGGSLRLSCATSGFTFTDYYLSWVRQPP |
| FR1-CDR1-FR2- | GKAFEWLGFIRNRADGYTTNYSASVKGRFTISRDNSQSILYL |
| CDR2-FR3-CDR3- | QMNTLRVEDSATYYCARDNDHYAMDYWGQGTSVTVSS |
| FR4 (SEQ ID NO: 17) | |
| VL | DVVMTQTPLSLSVTIGQPASISCKSSQSLLYSNGKTYLNWL |
| FR1-CDR1-FR2- | QQRPGQAPKHLMYQVSKLDPGIPDRFSGSGSETDFILKISRV |
| CDR2-FR3-CDR3- | EAEDLGIYYCLQGTYYPYTFGGGTKLEIK |
| FR4 (SEQ ID NO: 18) | |
| H-VD nucleic acid | gaggtgaggctggtggagtctggaggaggcttggtacagcctgggggttctctgagactctcct |
| sequence (SEQ ID | gtgcaacttctgggttcaccttcactgattactacctgagctgggtccgccagcctccaggaaag |
| NO: 19) | gcatttgagtggttgggttttattagaaacagagctgatggttacacaacaaactacagtgcatctg |
| | tgaagggtcggttcaccatctccagagataattcccaaagcatcctctatcttcaaatgaacaccc |
| | tgagagttgaggacagtgccacttattactgtgcaagagacaatgatcactatgctatggactact |
| | ggggtcaaggaacctcagtcaccgtctcttca |
| L-VD nucleic acid | gatgttgtgatgacccagactccactgtattgtcggttaccattggacaaccagcctctatctatg |
| sequence (SEQ ID | caagtcgagtcagagcctcttatatagtaatggaaagacatatttgaattggttacaacagaggcc |
| NO: 20) | tggccaggctccaaagcacctaatgtatcaggtgtccaaactggaccctggcatccctgacagg |
| | ttcagtggcagtggatcagaaacagattttatacttaaaattagcagagtggaggctgaagatttg |
| | ggaatttattactgcttgcaaggtacatattatccgtacacgttcggaggggggaccaagttggaa |
| | ataaaa |

The CDRs sequences of 2H2D11 and variants are in bold.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen (Ac-Met11 Tau species). As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. In the context of the invention, the amino acid residues of the antibody of the invention are numbered according to the IMGT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999); Lefranc, M.-P., Pommié, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003)). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. (http://www.imgt.org/IMGTScientificChart/Nomenclature/IMGT-FRCDRdefinition.html).

As used herein, the term “specificity” refers to the ability of an antibody to detectably bind an epitope presented on an antigen, such as AcMet11-Tau, while having relatively little detectable reactivity with non-Acetylated form Met11-Tau proteins or structures (such as other proteins expressed on TAM, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is AcMet11-Tau).

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The antibody of the invention may be assayed for specific binding by any method known in the art. Many different competitive binding assay format(s) can be used for epitope binding. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A immunoassays, and complement-fixation assays. Such assays are routine and well known in the art (see, e.g., Ausubel et al., eds, 1994 Current Protocols in Molecular Biology, Vol. 1, John Wiley & sons, Inc., New York). For example, the BIACORE® (GE Healthcare, Piscataway, N.J.) is one of a variety of surface plasmon resonance assay formats that are routinely used to epitope bin panels of monoclonal antibodies. Additionally, routine cross-blocking assays such as those described in Antibodies, A Laboratory Manual, Cold Spring Harbor Laboratory, Ed Harlow and David Lane, 1988, can be performed.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope.

As used herein, the percent identity between two sequences is a function of the number of identical positions shared by the sequences (i. e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Myers and W. Miller (Comput. Appl. Biosci. 4: 11-17, 1988) which has been incorporated into the ALIGN program. In addition, the percent identity between two amino acid sequences can be determined using the Needleman and Wunsch (J. Mol. Biol. 48:443-453, 1970) algorithm which has been incorporated into the GAP program in the GCG software package. Yet another program to determine percent identity is CLUSTAL (M. Larkin et al., Bioinformatics 23:2947-2948, 2007; first described by D. Higgins and P. Sharp, Gene 73:237-244, 1988) which is available as stand-alone program or via web servers (see http://www.clustal.org/).

The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

In some embodiment, the antibody of the invention comprises:

(a) a heavy chain wherein the variable domain comprises:
a H-CDR1 having at least 5, 4, 3, 2, 1 conservative substitutions within the H-CDR1 of antibody 2H2D11 (SEQ ID NO:11);
a H-CDR2 having at least 10, 9, 8, 7, 6, 5, 4, 3, 2, 1 conservative substitutions within the H-CDR2 of antibody 2H2D11 (SEQ ID NO:12;
a H-CDR3 having at least 9, 8, 7, 6, 5, 4, 3, 2, 1 conservative substitutions within the H-CDR3 of antibody 2H2D11 (SEQ ID NO:13);

(b) a light chain wherein the variable domain comprises:
a L-CDR1 having at least 10, 9, 8, 7, 6, 5, 4, 3, 2, 1 conservative substitutions within the L-CDR1 of antibody 2H2D11 (SEQ ID NO:14);
a L-CDR2 having at least 10, 9, 8, 7, 6, 5, 4, 3, 2, 1 conservative substitutions within the L-CDR2 of antibody 2H2D11 (SEQ ID NO:15);
a L-CDR3 having at least 10, 9, 8, 7, 6, 5, 4, 3, 2, 1 conservative substitutions within the L-CDR3 of antibody 2H2D11 (SEQ ID NO:16).

(c) that binds to AcMet11-Tau with substantially the same affinity as an antibody having a variable light chain domain (VL) and/or a variable heavy chain domain (VH) of the antibody 2H2D11.

The antibodies of the present invention are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In one embodiment, the monoclonal antibody of the invention is a chimeric antibody, particularly a chimeric mouse/human antibody.

According to the invention, the term “chimeric antibody” refers to an antibody which comprises a VH domain and a VL domain of a non-human antibody, and a CH domain and a CL domain of a human antibody.

In some embodiments, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig. and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204,244).

In another embodiment, the monoclonal antibody of the invention is a humanized antibody. In particular, in said humanized antibody, the variable domain comprises human acceptor frameworks regions, and optionally human constant domain where present, and non-human donor CDRs, such as mouse CDRs.

In another embodiment, the monoclonal antibody of the invention is a caninized or primatized based on the same methods of humanization.

According to the invention, the term “humanized antibody” refers to an antibody having variable region framework and constant regions from a human antibody but retains the CDRs of a previous non-human antibody.

The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

Fragments of the Antibody of the Present Invention

In one embodiment, the antibody of the invention is an antigen binding fragment selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL or VH chains as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:17 or SEQ ID NO:18.

The term “antigen binding fragment” of an antibody, as used herein, refers to one or more fragments of an intact antibody that retain the ability to specifically binds to a given antigen (e.g., AcMet11-Tau). Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilized by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

The Fab of the present invention can be obtained by treating an antibody which specifically reacts with AcMet11-Tau with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with AcMet11-Tau with a protease, pepsin. Also, the F(ab′)2 can be produced by binding Fab′ described below via a thioether bond or a disulfide bond.

The Fab′ of the present invention can be obtained treating F(ab′)2 which specifically reacts with AcMet11-Tau with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

Nucleic Acid Molecule, Vector, Host Cells

A further object of the invention relates to a nucleic acid molecule encoding an antibody according to the invention. More particularly the nucleic acid molecule encodes a heavy chain or a light chain of an antibody of the present invention.

In a particular embodiment, the nucleic acid molecule comprises a nucleic acid sequence having at least 70% of identity with SEQ ID NO:19 or SEQ ID NO:20.

More particularly the nucleic acid molecule comprises a nucleic acid sequence having at least 80% of identity with SEQ ID NO:19 or SEQ ID NO:20.

More particularly the nucleic acid molecule comprises a nucleic acid sequence having at least 90% of identity with SEQ ID NO:19 or SEQ ID NO:20.

More particularly the nucleic acid molecule comprises a nucleic acid sequence having at least 91, 92, 93, 94, 95, 96, 97, 98 or 99% of identity with SEQ ID NO:19 or SEQ ID NO:20.

Variable domain Heavy chain: nucleic acid sequence
FR1-CDR1-FR2-CDR2-FR3-CDR3-FR4:
SEQ ID NO: 19
GAGGTGAGGCTGGTGGAGTCTGGAGGAGGCTTGGTACAGCCTGGGGGTT
CTCTGAGACTCTCCTGTGCAACTTCTGGGTTCACCTTCACTGATTACTA
CCTGAGCTGGGTCCGCCAGCCTCCAGGAAAGGCATTTGAGTGGTTGGGT
TTTATTAGAAACAGAGCTGATGGTTACACAACAAACTACAGTGCATCTG
TGAAGGGTCGGTTCACCATCTCCAGAGATAATTCCCAAAGCATCCTCTA
TCTTCAAATGAACACCCTGAGAGTTGAGGACAGTGCCACTTATTACTGT
GCAAGAGACAATGATCACTATGCTATGGACTACTGGGGTCAAGGAACCT
CAGTCACCGTCTCTTCA
Variable domain Light chain: nucleic acid sequence
FR1-CDR1-FR2-CDR2-FR3-CDR3-FR4:
SEQ ID NO: 20
GATGTTGTGATGACCCAGACTCCACTGTCTTTGTCGGTTACCATTGGACA
ACCAGCCTCTATCTCTTGCAAGTCGAGTCAGAGCCTCTTATATAGTAATG
GAAAGACATATTTGAATTGGTTACAACAGAGGCCTGGCCAGGCTCCAAAG
CACCTAATGTATCAGGTGTCCAAACTGGACCCTGGCATCCCTGACAGGTT
CAGTGGCAGTGGATCAGAAACAGATTTTATACTTAAAATTAGCAGAGTGG
AGGCTGAAGATTTGGGAATTTATTACTGCTTGCAAGGTACATATTATCCG
TACACGTTCGGAGGGGGGACCAAGTTGGAAATAAAA

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. Nos. 6,013,516, 4,861,719, 5,278,056 and WO 94/19478.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like.

In some embodiments, the vector useful for the present invention is a viral vector. Gene delivery viral vectors useful in the practice of the present invention (for direct in vivo delivery of the antibody of the invention) can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction. Examples of viral vector include but are not limited to adenoviral, retroviral, lentiviral, herpesvirus and adeno-associated virus (AAV) vectors. In some embodiments, the vector of the present invention is an adeno-associated viral (AAV) vector. By an “AAV vector” is meant a vector derived from an adeno-associated virus serotype, including without limitation AAV1, AAV2, AAV3, AAV4, AA5, AAV6, AAV7, AAV8, AAV9, AAVrh10 or any other serotypes of AAV that can infect humans, monkeys or other species. In some embodiments, the AAV vector of the present invention is selected from vectors derived from AAV serotypes having tropism for and high transduction efficiencies in cells of the mammalian central and peripheral nervous system, particularly neurons, neuronal progenitors, astrocytes, oligodendrocytes and glial cells. In some embodiments, the AAV vector is an AAV4, AAV9 or an AAVrh10 that have been described to well transduce brain cells especially neurons.

The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention. Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

Functional Variants and Competitive Antibodies

The present invention provides antibodies comprising functional variants of the VL region, VH region, or one or more CDRs of the 2H2D11 antibody. A functional variant of a VL, VH, or CDR used in the context of a monoclonal antibody of the present invention still allows the antibody to retain at least a substantial proportion (at least about 50%, 60%, 70%, 80%, 90%, 95% or more) of the affinity/avidity and/or the specificity/selectivity of the parent antibody (i.e. 6-25 antibody) and in some cases such a monoclonal antibody of the present invention may be associated with greater affinity, selectivity and/or specificity than the parent Ab. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent Ab. The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:

Aliphatic residues I, L, V, and M

Cycloalkenyl-associated residues F, H, W, and Y

Hydrophobic residues A, C, F, G, H, I, L, M, R, T, V, W, and Y

Negatively charged residues D and E

Polar residues C, D, E, H, K, N, Q, R, S, and T

Positively charged residues H, K, and R

Small residues A, C, D, G, N, P, S, T, and V

Very small residues A, G, and S

Residues involved in turn A, C, D, E, G, H, K, N, Q, R, S, P, and formation T

Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of the 6-25 antibody. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 70% of identity to the parent peptide. According to the present invention a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with the second amino acid sequence. According to the present invention a first amino acid sequence having at least 90% of identity with a second amino acid sequence means that the first sequence has 90; 91; 92; 93; 94; 95; 96; 97; 98; 99; or 100% of identity with the second amino acid sequence.

In some embodiments, the antibody of the present invention is an antibody having a heavy chain comprising i) a H-CDR1 having at least 90; 91; 92; 93; 94; 95; 96; 97; 98; or 99% of identity with the H-CDR1 of the antibody of the invention, ii) a H-CDR2 having at least 90; 91; 92; 93; 94; 95; 96; 97; 98; or 99% of identity with the H-CDR2 of the antibody of the invention and iii) a H-CDR3 having at least 90; 91; 92; 93; 94; 95; 96; 97; 98; or 99% of identity with the H-CDR3 of the antibody of the invention.

In some embodiments, the antibody of the present invention is an antibody having a light chain comprising i) a L-CDR1 having at least 90; 91; 92; 93; 94; 95; 96; 97; 98; or 99% of identity with the L-CDR1 of the antibody of the invention, ii) a L-CDR2 having at least 90; 91; 92; 93; 94; 95; 96; 97; 98; or 99% of identity with the L-CDR2 of the antibody of the invention and iii) a L-CDR3 having at least 90; 91; 92; 93; 94; 95; 96; 97; 98; or 99% of identity with the L-CDR3 of the antibody of the invention.

In some embodiments, the antibody of the present invention is an antibody having a heavy chain comprising i) a H-CDR1 having at least 90; 91; 92; 93; 94; 95; 96; 97; 98; or 99% of identity with the H-CDR1 of the antibody of the invention, ii) a H-CDR2 having at least 90; 91; 92; 93; 94; 95; 96; 97; 98; or 99% of identity with the H-CDR2 of the antibody of the invention and iii) a H-CDR3 having at least 90; 91; 92; 93; 94; 95; 96; 97; 98; or 99% of identity with the H-CDR3 of the antibody of the invention and a light chain comprising i) a L-CDR1 having at least 90; 91; 92; 93; 94; 95; 96; 97; 98; or 99% of identity with the L-CDR1 of the antibody of the invention, ii) a L-CDR2 having at least 90; 91; 92; 93; 94; 95; 96; 97; 98; or 99% of identity with the L-CDR2 of the antibody of the invention and iii) a L-CDR3 having at least 90; 91; 92; 93; 94; 95; 96; 97; 98; or 99% of identity with the L-CDR3 of the antibody of the invention.

In some embodiments, the antibody of the present invention is an antibody having a heavy chain comprising i) the H-CDR1 of the antibody of the invention, ii) the H-CDR2 of the antibody of the invention and iii) the H-CDR3 of the antibody of the invention.

In some embodiments, the antibody of the present invention is an antibody having a light chain comprising i) the L-CDR1 of the antibody of the invention, ii) the L-CDR2 of the antibody of the invention and iii) the L-CDR3 of the antibody of the invention.

In some embodiments, the antibody of the present invention is an antibody having a heavy chain comprising i) the H-CDR1 of the antibody of the invention, ii) the H-CDR2 of the antibody of the invention and iii) the H-CDR3 of the antibody of the invention and a light chain comprising i) the L-CDR1 of the antibody of the invention, ii) the L-CDR2 of the antibody of the invention and iii) the L-CDR3 of the antibody of the invention.

In some embodiments, the antibody of the present invention is an antibody having a heavy chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:17.

In some embodiments, the antibody of the present invention is an antibody having a light chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:18.

In some embodiments, the antibody of the present invention is an antibody having a heavy chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:17 and a light chain having at least 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with SEQ ID NO:18.

In some embodiments, the antibody of the present invention is an antibody having a heavy chain which is identical to SEQ ID NO:17.

In some embodiments, the antibody of the present invention is an antibody having a light chain identical to SEQ ID NO:18.

In some embodiments, the antibody of the present invention is an antibody having a heavy chain identical to SEQ ID NO:17 and a light chain identical to SEQ ID NO:18.

In another aspect, the invention provides an antibody that competes for binding to AcMet11-Tau with the antibody of the invention.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscataway, N.J.) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Antibody Engineering

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell—epitopes to thereby reduce the potential immunogenicity of the antibody. This approach is also referred to as “deimmunization” and is described in further detail in U.S. Patent Publication No. 20030153043 by Carr et al.

In some embodiments, the glycosylation of an antibody is modified. Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

In some embodiments, some mutations are made to the amino acids localized in aggregation “hotspots” within and near the first CDR (CDR1) to decrease the antibodies susceptibility to aggregation (see Joseph M. Perchiacca et al., Proteins 2011; 79:2637-2647).

The antibody of the present invention may be of any isotype. The choice of isotype typically will be guided by the desired effector functions. IgG1 and IgG3 are isotypes that mediate such effectors functions as ADCC or CDC, when IgG2 and IgG4 don't or in a lower manner. Either of the light chain constant regions, kappa or lambda, may be used. If desired, the class of a monoclonal antibody of the present invention may be switched by known methods. Typical, class switching techniques may be used to convert one IgG subclass to another, for instance from IgG1 to IgG2. Thus, the effector function of the monoclonal antibodies of the present invention may be changed by isotype switching to, e.g., an IgG1, IgG2, IgG3, IgG4, IgD, IgA, IgE, or IgM antibody for various therapeutic uses.

In some embodiments, the antibody of the present invention is a full-length antibody. In some embodiments, the full-length antibody is an IgG2 antibody. In some embodiments, the full-length antibody is an IgG4 antibody.

In some embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In some embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the complement. This approach is described in further detail in U.S. Pat. Nos. 5,624,821 and 5,648,260, both by Winter et al.

Half Life

In one embodiment, the antibody is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Antibodies with increased half lives and improved binding to the neonatal Fc receptor (FcRn), which is responsible for the transfer of maternal IgGs to the foetus (Guyer et al., J. Immunol. 117:587 (1976) and Kim et al., J. immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitutions of Fc region residue 434 (U.S. Pat. No. 7,371,826).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the antibodies that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polysialylation is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

Others Modifications

In certain embodiments of the invention the antibodies have been engineered to increase pI (isoelectric point) and improve their drug-like properties. The pI of a protein is a key determinant of the overall biophysical properties of a molecule. Antibodies that have low pIs have been known to be less soluble, less stable, and prone to aggregation. Further, the purification of antibodies with low pI is challenging and can be problematic especially during scale-up for clinical use. Increasing the pI of the antibodies of the invention or fragments thereof improved their solubility, enabling the antibodies to be formulated at higher concentrations (>100 mg/ml). Formulation of the antibodies at high concentrations (e.g. >100 mg/ml) offers the advantage of being able to administer higher doses of the antibodies into eyes of patients via intravitreal injections, which in turn may enable reduced dosing frequency, a significant advantage for treatment of chronic diseases including neurodegenerative disorders like tauopathies. Higher pIs may also increase the FcRn-mediated recycling of the IgG version of the antibody thus enabling the drug to persist in the body for a longer duration, requiring fewer injections. Finally, the overall stability of the antibodies is significantly improved due to the higher pI resulting in longer shelf-life and bioactivity in vivo. Preferably, the pI is greater than or equal to 8.2.

Vaccine Composition

As inventors discovered the new Tau species AcMet11-Tau involved in Tau pathology development, thus opening the way to an innovative vaccination approach.

Accordingly, another object of the invention is a vaccine composition comprising a polypeptide of following amino acid sequence (N-α-acetyl)MEDHAGTYGLG (SEQ ID NO:8) and an immunoadjuvant compound.

By “vaccine composition” it is herein intended a substance which is able to induce an immune response in an individual, and for example to induce the production of antibodies directed against the AcMet11-Tau species.

A vaccine is defined herein as a biological agent which is capable of providing a protective response in an animal to which the vaccine has been delivered and is incapable of causing severe disease. The vaccine stimulates antibody production or cellular immunity against the pathogen causing the disease; administration of the vaccine thus results in immunity from the disease.

Preferably, said immunoadjuvant compound is selected in the group consisting of Freund complete adjuvant, Freund incomplete adjuvant, aluminium hydroxide, and calcium phosphate.

In particular, said antigenic polypeptide, can have the following formula (I):

NH2-PepNt-[(I)n-PepXn]n-PepCt-COOH (I),

wherein:
“PepNt” consists of a polypeptide having an amino acid length varying from 0 to 100 amino acid residues and located at the N-terminal end of the polypeptide of formula (I);
“[(I)n-PepXn]” consists of a polypeptide unit wherein:
“(I)1” to-“(I)n” each consists of, one independently from each other, a polypeptide of following amino acid sequences (N-α-acetyl)MEDHAGTYGLG (SEQ ID NO:8) “with n being an integer from 1 to 12; and
“PepX1” to “PepXn” each consists of, one independently from the other, a spacer polypeptide having an amino acid length varying from 0 to 30 amino acid residues, with n being an integer from 1 to 12; and
n is the number of [(I)n-PepXn] polypeptide units in said polypeptide, with n being an integer from 1 to 12; and
“PepCt” consists of a polypeptide having an amino acid length varying from 0 to 100 amino acid residues and located at the C-terminal end of the polypeptide of formula (I).

In particular, said antigenic polypeptide, can have the following amino acid sequence (N-α-acetyl)MEDHAGTYGLG (SEQ ID NO:8) or the following formula (I) as defined above.

More particularly, said antigenic polypeptide, comprising or consisting:

(i) the amino acids sequence consisting of Tau N-Alpha Acetyl-Met11-352 (SEQ ID NO:1);

(ii) the amino acids sequence consisting of Tau N-Alpha Acetyl-Met11-381 (SEQ ID NO:2);

(iii) the amino acids sequence consisting of Tau N-Alpha Acetyl-Met11-383 (SEQ ID NO:3)

(iv) the amino acids sequence consisting of Tau N-Alpha Acetyl-Met11-410 (SEQ ID NO:4)

(v) the amino acids sequence consisting of Tau N-Alpha Acetyl-Met11-412 (SEQ ID NO:5);

(vi) the amino acids sequence consisting of Tau N-Alpha Acetyl-Met11-441 (SEQ ID NO:6));

(vii) the amino acids sequence consisting of Tau N-Alpha Acetyl-Met11-776 (SEQ ID NO:7

(viii) a fragment of at least 9 consecutive amino acids starting from the N-Alpha Acetyl methionine residue at position 11 of the sequence of (i), to (vii)

(ix) an amino acid sequence substantially homologous to the sequence of (i), to (viii) preferably an amino acid a sequence at least 80% identical to the sequence of (i) to (viii).

A peptide “substantially homologous” to a reference peptide may derive from the reference sequence by one or more conservative substitutions. Two amino acid sequences are “substantially homologous” or “substantially similar” when one or more amino acid residue are replaced by a biologically similar residue or when greater than 80% of the amino acids are identical, or greater than about 90%, preferably greater than about 95%, are similar (functionally identical). Preferably, the similar, identical or homologous sequences are identified by alignment using, for example, the GCG (Genetics Computer Group, Program Manual for the GCG Package, Version 7, Madison, Wis.) pileup program, or any of the programs known in the art (BLAST, FASTA, etc.). The percentage of identity may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

The term “conservative substitution” as used herein denotes the replacement of an amino acid residue by another, without altering the overall conformation and function of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Neutral hydrophilic amino acids, which can be substituted for one another, include asparagine, glutamine, serine and threonine.

By “substituted” or “modified” the present invention includes those amino acids that have been altered or modified from naturally occurring amino acids.

As such, it should be understood that in the context of the present invention, a conservative substitution is recognized in the art as a substitution of one amino acid for another amino acid that has similar properties.

According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; or 99% of identity with the second amino acid sequence. Amino acid sequence identity is preferably determined using a suitable sequence alignment algorithm and default parameters, such as BLAST P (Karlin and Altschul, 1990).

Said antigenic peptide can be covalently linked through an amino acid residue to a carrier protein or a synthetic polymer.

In order to enhance peptide immunogenicity, the peptide of formula (I) can be covalently linked (“conjugated”) to a larger molecule which serves as a carrier.

Attachment of the peptide to the carrier can be by one of several methods, including linking through a peptide Lys using glutaraldehyde (Reichlin, Methods Enzymol. 70: 159-165, 1980) or DCC procedures (for example, Atassi et al., Biochem. Biophys. Acta 670: 300-302, 1981), through a Peptide Asp or Glu using DCC (Bauminger et al., Methods Enzymol 70: 151-159, 1980), through a peptide Tyr using bis-diazotized benzidine (Walter et al., Proc. Nat. Acad. Sci. USA 77: 5197-5200, 1980), through photochemical attachment sites (Parker et al., Cold Spring Harbor Symposium—Modern Approaches to Vaccines, Ed. Chanock & Lerner, Cold Spring Harbor Press, New York, 1984), or through a peptide Cys (Liu et al., Biochem. 18: 690-697, 1979).

Peptide carrier conjugates can be separated from excess free peptide by dialysis or gel filtration. The level of loading of the peptide on the carrier can be determined either using a radioactive tracer to establish the loading level in a particular procedure, or by quantitative amino acid analysis of the conjugate, in comparison with the unloaded carrier. It is convenient, when using the latter technique, to incorporate a unique non-natural amino acid into the peptide, at the N-terminal or C-terminal side, such as Me, which can then serve as a quantitative marker for peptide incorporation, as measured by amino acid analysis of the conjugate. This Nle can also function as a spacer between the antigenic site and any amino acid incorporated to facilitate attachment, such as Cys, Lys, or Tyr, as described above.

Preferably, said carrier protein is selected from the group consisting of keyhole limpet hemocyanin (KLH), bovine serum albumin, or diphtheria toxoid.

In a vaccine composition according to the invention, said synthetic polymer can be a multiple branch peptide construction comprising a core matrix comprised of lysine residues.

Radially branched systems using lysine skeletons in polymers have been used by J. P. Tam [Proc. Natl. Acad. Sci. U.S.A., 85, 5409-5413 (1988)] to develop antigens without the use of carriers. Those antigens were designed to generate vaccines against a variety of diseases. Specifically, antigens for generating vaccines against malaria disease are described in PCT patent application ser. no. WO93/10152, WO2006/029887, WO2007/003384, WO2009/021931 and WO2009/080715, WO2015038708

The core matrix is preferably a dendritic polymer which is branched in nature, preferably with each of the branches thereof being identical. The core matrix is based on a core molecule which has at least two functional groups to which molecular branches having terminal functional groups are covalently bonded. Exemplary for use to form the core matrix is lysine. A central lysine residue is bonded to two lysine residues, each through its carboxyl group, to one of the amino groups of the central lysine residue. This provides a molecule with four amino groups, which may be a core matrix for a structure comprising four peptides of formula (I). The manufacture of the above structures, is known in the art. See, e.g., Tam et al., J. Immun. 148, 914-920 (1992) and Wang et al., Science, 254, 285-288 (1991).

Additionally, spacers between said peptide and said carrier protein or synthetic polymer can be added. A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The invention concerns also a vaccine composition comprising a peptide comprising the amino acid sequence (N-α-acetyl)MEDHAGTYGLG (SEQ ID NO:8).

said peptide, being covalently linked through an amino acid residue to a carrier protein or to a synthetic polymer.

Preferably, said carrier protein is selected from the group consisting of keyhole limpet hemocyanin (KLH), bovine serum albumin, or diphtheria toxoid.

The synthetic polymer can be a multiple branch peptide construction comprising a core matrix comprised of lysine residues. Spacers between said polypeptide and said carrier protein or synthetic polymer can be introduced.

Preferably, in the vaccine composition cited immediately above, there are spacers between said polypeptide and said carrier protein or synthetic polymer.

Therapeutic Uses

As described in the experimental section, the antibody of the present invention targets AcMet11-Tau polypeptides, which are involved in Tau pathology development. Inventors have shown that AcMet11-Tau species potentiate Tau pathology development in Thy-Tau Transgenic mice (FIGS. 3 and 4), and are involved in pathological process, at least by accelerating Tau pathology.

Antibodies, fragments or immunoconjugates of the invention may be useful for treating Tauopathies. The antibodies of the invention may be used alone or in combination with any suitable agent.

In each of the embodiments of the treatment methods described herein, the antibody of the invention or antibody-drug conjugate of the invention is delivered in a manner consistent with conventional methodologies associated with management of the disease or disorder for which treatment is sought. In accordance with the disclosure herein, an effective amount of the antibody or antibody-drug conjugate is administered to a patient in need of such treatment for a time and under conditions sufficient to prevent or treat the disease or disorder.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “therapeutically effective amount” or “effective amount” refers to an amount effective, at dosages and for periods of time necessary, to achieve a desired therapeutic result. A therapeutically effective amount of the antibody of the present invention may vary according to factors such as the disease state, age, sex, and weight of the individual, and the ability of the antibody of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the antibody of the present invention depend on the disease or condition to be treated and may be determined by the persons skilled in the art. A physician having ordinary skill in the art may readily determine and prescribe the effective amount of the pharmaceutical composition required. For example, the physician could start doses of the antibody of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit tauopathies may, for example, be evaluated in an animal model system predictive of efficacy in human tauopathies. Alternatively, this property of a composition may be evaluated by examining the ability of the compound to induce neuroprotection by in vitro assays known to the skilled practitioner. A therapeutically effective amount of a therapeutic compound may decrease the neurofibrillary degeneration, or otherwise ameliorate cognitive decline symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, one object of the present invention relates to a method of treating tauopathies in a subject in need thereof comprising administering to the subject a therapeutically effective amount of an antibody of the present invention.

In another aspect, the present invention relates to the antibody of the present invention, as defined in any aspect or embodiment herein, for use as a medicament.

In another aspect, the present invention relates to the use of the antibody of the present invention for the treatment of tauopathy.

The term “tauopathy” has its general meaning in the art and refers to a disease characterized by Tau aggregation (Iqbal, K. et al. Biochimica et Biophysica Acta (BBA) 1739 (2005) 198-210). Tauopathies include among others, Alzheimer Disease, Down syndrome; Guam parkinsonism dementia complex; Dementia pugilistica and other chronic traumatic encephalopathies; myotonic dystrophies; Niemann-Pick disease type C; Pick disease; argyrophilic grain disease; Fronto-temporal dementia; Cortico-basal degeneration; Pallido-ponto-nigral degeneration; Progressive supranuclear palsy; and Prion disorders such as Gerstmann-Sträussler-Scheinker disease with tangles.

In a particular embodiment, the Tauopathy disorders is Alzheimer Disease.

Pharmaceutical Compositions

An aspect of the present invention relates to a pharmaceutical composition comprising the antibody of the invention.

Typically, the antibody of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m2 and 500 mg/m2. However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of an anti-Tau antibody of the invention.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

### FIGURES

FIG. 1. AcMet11-Tau is a pathological Tau species found in insoluble aggregated Tau fraction of AD brains. A) Representative Western blot analysis of human parietal cortex fractions, using AT100 antibody that labels aggregated Tau proteins present in Sarkosyl insoluble fraction of AD brains (Insoluble). B) Sandwich ELISA showing that likewise hyperphosphorylated Tau (pSer396), AcMet11-Tau species is detected in insoluble fraction of AD brains, containing aggregated Tau proteins (analysis using fractions from a pool of parietal cortex from 4 AD patients with braak V-VI staging).

FIG. 2. AcMet11-Tau is an early event in Tau pathology (detected as early as 2 months in hippocampus of Thy-Tau22 transgenic mice. 2H2D11 antibody-based ELISA in hippocampus of Thy-Tau22 and their littermate WT mice of 2, 3 and 6 months of age (*** p<0.0001; one-Way ANOVA followed by LSD Fisher test).

FIG. 3. Validation of lentiviral vectors batches in primary culture of neurons prior to in vivo studies. Western Blot analysis showing the same level of Tau expression from lentiviral vectors coding for full-length Tau (LV-Tau-FL), and Met11-Tau (LVMet11-Tau); Met11-Tau is detected as N-α-acetylated form in primary culture of neurons (2H2D11 antibody immunoblotting). DIV: days in vitro.

FIG. 4. AcMet11-Tau/Met11-Tau species potentiate Tau pathology development in Thy-Tau30 transgenic mice as showed by the increase in the number of NFT bearing neurons: AT100 positive immunostaining. A) Thy-Tau30 transgenic mice (n=5/group) were injected with lentival vectors (LV), into CA1 region of the hippocampus, with respect to the bregma at the indicated coordinates (AP: antero-posterior; ML: medial-lateral; DV: dorso-ventral). The groups have been designed in order to discriminate between the effect related to human Tau expression (LV FL-Tau versus PBS) and the proper effect of Met11-Tau species, i.e., consequences of the outermost N-terminus deletion of Tau and/or the Met11 N-α-acetylation (LV Met11-Tau versus LV FL-Tau). Mice were injected at 1 month of age and scarified 2 months later. B) Immunohistochemical analysis of coronal brain sections, 2 months post-injections, using AT8 antibody that labels neurons bearing hyperphosphorylated Tau species (arrowhead) in CA1 region of hippocampus. C) Quantification of the mean number of neurons bearing neurofibrillary degeneration, labeled with AT100 antibody. For each animal, 4-6 coronal sections including the hippocampus were analyzed (n=5 mice/group). *p<0.05 one-Way ANOVA followed by a post-hoc Fisher's.

FIG. 5. Experimental design of passive immunotherapy against AcMet11-Tau in Thy-Tau22 mice: study 1. Repeated intraperitoneal injections (IP) of the monoclonal antibody directed against AcMet11-Tau (2H2D11 antibody; IgG2a isotype), and a non-specific antibody (IgG2a isotype control (purified from B69 hybridoma (ATCC® HB-9437™)) were performed in heterozygous Thy-Tau22 males and their littermate WT mice (n=2-3 mice/group; 10 mg antibody/kg). In the same experimental procedure, two mice groups were injected with PBS. Mice have received their first IP at 3 months of age and then every 10 days until 7 months of age. Mice received a last injection one week before sacrifice (at 8 months of age). Animals were sacrificed by cervical dislocation, brains removed. The right hemispheres were post-fixed for 7 days in 4% paraformaldehyde, then incubated in 20% sucrose for 24 hours and kept frozen at −80° C. until use for immunohistochemical analyses of Tau pathology. The left hemispheres were used to dissect out hippocampus by using a coronal acrylic slicer (Delta Microscopies) at 4° C. and stored at −80° C. for biochemical analyses of Tau pathology. Blood samples (collected at tail vein) were recovered prior to each IP injection for ELISA analyses of antibodies stability (titer).

FIG. 6. AcMet11-Tau peptide-based ELISA shows 2H2D11 antibody is stable and functional in mice blood over the repeated IP injections. Mice blood samples (SO to S7) recovered before each injection over immunization protocol (FIG. 5) were analyzed by Indirect ELISA (diluted 1:600) after coating with either the specific AcMet11-peptide or the negative control Tau-peptide. Antibody titers were determined thanks to the purified 2H2D11 antibody used as standard.

FIG. 7A. 2H2D11 antibody-based immunotherapy reduces MC1 immunostaining (Tau oligomers). Representative Immunohistochemical analysis of coronal hippocampus sections, using MC1 antibody, that labels Tau oligomers species. Histograms showed quantification of the MC1 staining as % of CA1 area. For each animal, 4-6 coronal sections including the hippocampus were analyzed (n=3 mice/group); ** p=0.0033 unpaired Student's t-test. 7B. 2H2D11 antibody-based immunotherapy reduces the number of AT100 positive neurons (neurons bearing neurofibrillary tangles). Representative Immunohistochemical analysis of coronal hippocampus sections, using AT100 antibody, that labels Tau aggregates species. Histograms showed quantification of the mean number of neurons bearing neurofibrillary degeneration (AT100 positive neurons) in the hippocampus, averaged per mm2. For each animal, 4 to 6 coronal sections including the hippocampus were analyzed (n=3 mice/group); * p=0,014 unpaired Student's t-test.

FIG. 8. 2H2D11 antibody-based immunotherapy reduces insoluble Tau aggregates. Western blot analysis of sarkosyl soluble (S) and insoluble (P) fractions from mice hippocampus: A) using AT100 antibody that labels aggregated Tau proteins present in Sarkosyl insoluble fraction (Insoluble); B) using HT7 antibody (pan Tau antibody); C) Densitometry quantification of western Blot for HT7 and schematic representation of the % of insoluble Tau proteins.

FIG. 9. Experimental design of passive immunotherapy against AcMet11-Tau in Thy-Tau22 mice: study 2. Repeated intraperitoneal injections (IP) of the monoclonal antibody directed against AcMet11-Tau (2H2D11 antibody; IgG2a isotype) as well as with a non-specific antibody (IgG2a isotype control (purified from B69 hybridoma (ATCC® HB-9437™)) were performed in heterozygous Thy-Tau22 males (n=15 mice/group; 10 mg antibody/kg). In the same experimental procedure, littermate WT mice group were injected with either PBS (n=9) or IgG2a isotype control (n=6), to get a baseline in behavioral evaluations. Experimental groups made of females were also included.

Mice have received their first IP at 3 months of age and then every 2 weeks until behavioral evaluation at 7 months of age. Mice received a last injection one week before sacrifice (at 8 months of age). Animals were sacrificed by cervical dislocation, brains removed. The right hemispheres were post-fixed for 7 days in 4% paraformaldehyde, then incubated in 20% sucrose for 24 hours and kept frozen at −80° C. until use. The left hemispheres were used to dissect out hippocampus and cortex by using a coronal acrylic slicer (Delta Microscopies) at 4° C. and stored at −80° C. for biochemical analyses.

Blood samples (collected at tail vein) were recovered, prior to the first injection as well as at the middle and the end of the immunization protocol, for ELISA analyses.

FIG. 10. Passive immunotherapy against AcMet11-Tau improves spatial memory of Thy-Tau22 mice (Y-Maze). WT control animals exhibited a preference for the novel over the other familiar arm (** p<0.01 N vs. O; One-Way ANOVA followed by LSD Fisher test). As expected, THY-Tau22 treated with IgG2a isotype control did not display proper spatial memory as characterized by an absence of preference for the novel vs. the other arm (p=0.21, N vs. O) and a percentage of time spent in the novel arm significantly reduced as compared to WT animals (# p<0.05; One-Way ANOVA followed by LSD Fisher test). Conversely, THY-Tau22 treated with the 2H2D11 antibody exhibited a significant preference for the new over the other arm (** p<0.01 N vs. O; One-Way ANOVA followed by LSD Fisher test) together with a percentage of time spent in the novel arm significantly enhanced as compared to THY-Tau22 animals treated with the IgG2a isotype control antibody (° p<0.05; One-Way ANOVA followed by LSD Fisher test), supporting restored spatial memory abilities.

FIG. 11: 2C12C8 hybridoma supernatant allows specific detection of N-alpha-terminally acetylated Met11-Tau peptide by indirect ELISA. Histograms indicated representative ELISA OD values obtained by serial dilutions of supernatant from 2C12C8 hybridoma culture. As positive controls, we have used purified hTauE1 antibody (total Tau, 12-21) that similarly recognizes the 3 peptides we have used in indirect ELISA, and purified 2H2D11 antibody to show the specificity towards AcMet11-Tau peptide. The 3 peptides were described previously (WO 2018/178078): AcMet11-Tau peptide: N-alpha-acetyl-Met11-Tau peptide; Met11-Tau peptide: non-alpha acetylated truncated Met11 Tau; FL-Tau peptide: Tau peptide starting at Methionine1 and containing non-free Met11 Tau.

FIG. 12: Detection of N-terminally acetylated Met11-Tau calibrator by sandwich ELISA based on two distinct antibodies: 2C12C8 and 2H2D11. Top panel. Schematic representation of sandwich ELISA assay for detection of N-alpha-acetyl-Met11-Tau species; capture antibody was made of purified 2C12C8 or 2H2D11; detection antibody was made of TauE1 antibody (total Tau, 23-40). Bottom panel. For each antibody (2C12C8 and 2H2D11), the standard curve was made by using serial dilutions of an N-alpha-acetyled-Met11-Tau calibrator peptide

FIG. 13: Specific detection by Western Blot analysis of N-terminally acetylated Met11-Tau protein in cell lysates, using two distinct monoclonal antibodies: 2C12C8 and 2H2D11. Representative Western blot analyses of 10 μg of protein extracts from SH-SY5Y inducible cell lines overexpressing either Full-length Tau (Tau-412) or AcMet11-Tau (Met11-Tau), after 48 h of tetracycline treatment. Analysis was first performed using either 2H2D11 antibody or 2C12C8 antibody. Membranes were then re-probed with Tau C-ter antibody to display Tau protein expression in both cell lines.

FIG. 14: Specific detection of N-terminally acetylated Met11-Tau proteins by 2C12C8 antibody in the brain samples (hippocampus) from Alzheimer's disease patients (A) and Thy-Tau22 transgenic mice (B). A. Protein extracts from hippocampus of age-matched controls (n=6) and AD cases (n=7, from Braak IV-VI) were used in 2C12C8 antibody-based sandwich ELISA. Data showed that 2C12C8 antibody specifically reacted in AD samples (****p<0.0001; compared with unpaired t test). B. 2C12C8 antibody-based sandwich ELISA in hippocampus of Thy-Tau22 and their littermate WT mice of 3 and 7 months of age, (****p=0.0016; compared with unpaired t test).

FIG. 15: 2C12C8 antibody labels neurons displaying neurofibrillary degeneration in the hippocampus of Thy-Tau22 transgenic mice. Representative immunohistochemistry analyses of mouse sagittal brain sections, using 2C12C8 antibody. Panel A: section from Wt littermate mouse of 8 months of age; Panels B-C sections from Thy-Tau22 mice of 3, 8 and 13 months of age, respectively. Arrows showed some of the typical neurons with neurofibrillary degeneration features, displayed by 2C12C8 antibody.

### EXAMPLE 1

Materials and Methods

Generation of 2H2D11 antibody. 2H2D11 antibody was generated by immunization with an N-alpha-terminal acetyl Tau peptide (Ac-Met11-Tau peptide: {Nα-acetyl}MEDHAGTYGLG: SEQ ID No 8) corresponding to the Tau sequence from Methionine at position 11 to Glycine at position 21. This sequence is encoded by exon1; shared by all Tau isoforms. A cysteine residue has been added to the C-terminus of Ac-Met11-Tau peptide for KLH Conjugation. Balb/c mice were immunized subcutaneously with 3 boosts at days 14, 45 and 63. The lymphocytes from the spleen of the mouse displaying the highest titer were then fused with NS1 myeloma cells, according to the method described in (Pandey, 2010). The hybridoma supernatants were screened in indirect ELISA against the following different peptides:

Ac-Met11-Tau peptide: {Nα-acetyl}MEDHAGTYGLG; (SEQ ID No 8) the same sequence than Tau fragment used as antigen.

Met11-Tau peptide: MEDHAGTYGLG; (SEQ ID No 9)

Ac-Met1-Tau peptide: {Nα-acetyl}MAEPRQEFEVMEDHAGTYGLG (SEQ ID No 10); the peptide starts at Methionine 1 of Tau harboring an N-alpha-terminal acetylation.

Indirect ELISA screening of hybridoma supernatants allowed selection of a set of clones that specifically detect the Ac-Met11-Tau species with a slight or without any cross-reactivity with the free non-N-alpha-terminally-acetylated Methionine 11, nor with the non-truncated Methionine 11, nor with any N-alpha-acetyl-Methionine when it is not in the same amino acid context than Methionine11. Isotype and the type of light chain have been determined for the selected hybridoma (Table 2, Bellow).

TABLE 2

| Hybridoma designation | Isotype | light chain |
|---|---|---|
| 1C10 | IgG1/IgM | Kappa/Lannda |
| 2H2 | IgG2a | Kappa |
| 3F2 | IgG2A/IgM | Kappa/Lannda |
| 2C12 | IgG1 | Kappa |
| 9H4 | IgG2a | Kappa |

The hybridoma 2H2 was further subcloned and we selected the hybridoma clone that produces 2H2D11 antibody. The specificity of 2H2D11 antibody towards N-alpha-terminally acetylated methionine11 of Tau protein was reproducibly validated by indirect ELISA, western blotting and immunohistochemistry. The VH and VL sequences of 2H2D11 are provided.

Human Tissue Samples.

Human brain autopsy samples (parietal cortex) were from the Lille NeuroBank collection (Centre de Ressources Biologiques du CHU de Lille). Informed consent was obtained from all subjects. The Lille NeuroBank has been declared to the French Research Ministry by the Lille University Hospital (CHU-Lille) on Aug. 14, 2008 under the reference DC-2000-642 and fulfills the criteria defined by French Law regarding biological resources, including informed consent, ethics review committee approval and data protection. The ethical review committee of Lille NeuroBank approved the study. The stages of Tau pathology were categorized on the basis of neuropathological characteristics according to Braak et al., (2011).

Mice.

Thy-Tau transgenic mice lines (Thy-Tau22 and Thy-Tau30), of C57Bl6/J background, that develop with age neurofibrillary degeneration and memory deficits were generated by the overexpression of a human Tau isoform (1N4R) bearing two pro-aggregative mutations (G272V & P301S) under the control of the Thy1.2 neuronal promoter (Schindowski et al. 2006). Additional description is provided in Van der Jeugd et al. 2013 and Laurent et al. 2017, for Thy-Tau 22 model; and Leroy et al. 2007, for Thy-Tau 30 model.

Transgenic mice and littermate controls (WT) were housed in a pathogen-free facility, per cage (Techniplast cages 1284 L), with ad libitum access to food and water and maintained on a 12 h light/12 h dark cycle. The animals were maintained in compliance with European standards for the care and use of laboratory animals and all experimental protocols have obtained ethic approval: Agreement for experimentations on vertebrate animals (no 2015101320441671/2016-2020, from CEEA75, Lille, France). Agreement for genetically modified organism manipulation (OGM 2015-2020, No 1285, le Haut Conseil des Biotechnologies).

Lentiviral Vectors.

Lentiviral vectors (LV) with neuronal tropism (Deglon et al., 2000), carrying cDNA for Full-length Tau (Tau-412) and Met11-Tau (in the context of 4R isoform) were generated and produced and their titer determined as previously described (Caillierez et al., 2013).

Primary Neuronal Culture.

Primary cortical neurons were obtained from 15- to 17-day-old mouse embryos and prepared as follows. Briefly, Cortex was carefully dissected out and mechanically dissociated in culture medium by trituration with a polished Pasteur pipette. Once dissociated and counted, cells were plated in 6-well plates (800000 cells per well). For dissociation, plating and culture, Neurobasal medium supplemented with 2% B27, 500 μM glutamine and 1% antibiotic-antimycotic agent (Gibco, France) was used. Cells were maintained in a 5% CO2 humidified incubator at 37° C.

Lentiviral vectors-based infections were performed at DIV11; 400 ng of LVs were added per well. Three days later, the cells were washed once with phosphate-buffered saline and recovered in lysis buffer for WB analysis.

Stereotaxic Injections.

One-month-old heterozygous Thy-Tau30 transgenic mice were anesthetized with intraperitoneal injection of Ketamine (100 mg/kg) and Xylazine (20 mg/kg) mix. The animals were positioned on a stereotactic device (David Kopf Instrument) and bilateral injections were performed into CA1 region of the hippocampus, at the following coordinates with respect to the bregma: antero-posterior −2.5 mm, medial-lateral −1 mm (right side) and +1 (left side) and dorso-ventral −1.8 mm. Equivalent amounts of Lentiviral vectors (445 ng of p24) or PBS (2.5 μl) were injected at a rate of 0.25 μl/min, using a 10 μl glass syringe with a fixed needle (Hamilton; Dutscher, Brumath, France). Two months post-injections, mice were deeply anesthetized with pentobarbital sodium (50 mg/kg, i.p.), then transcardially perfused, first with cold NaCl (0.9%) and then with 4% paraformaldehyde in 0.1 mol/L phosphate-buffered saline (pH 7.4) during 20 min. Brains were post-fixed during 1 day in 4% paraformaldehyde, then incubated in 20% sucrose for 24 hours, frozen 1 min in Isopentane at −40° C. and then kept at −80° C. until use.

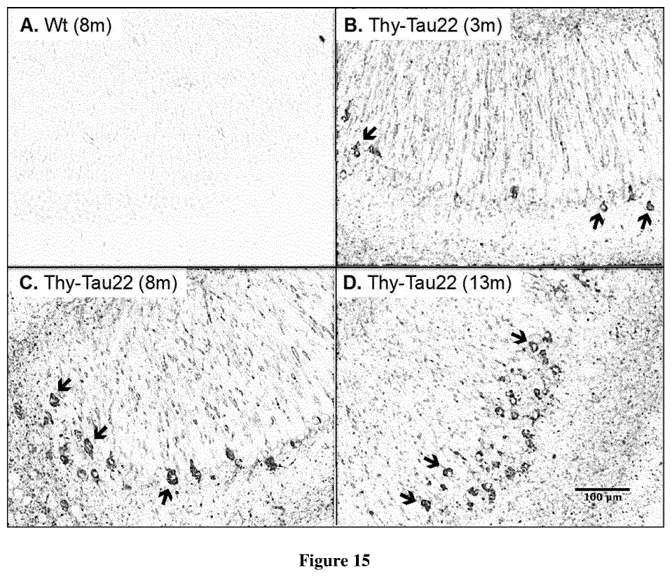

Passive Immunotherapy (FIG. 5 and FIG. 9).

Repeated intraperitoneal injections (IP) of the monoclonal antibody directed against AcMet11-Tau (2H2D11 antibody; IgG2a isotype) as well as with a non-specific antibody (IgG2a isotype control (purified from B69 hybridoma (ATCC® HB-9437™)) were performed in heterozygous Thy-Tau22 (10 mg antibody/kg). In the same experimental procedure, littermate WT mice group were injected with either PBS or IgG2a isotype control. Mice have received their first IP at 3 months of age and then every 10 days (for study 1) or every 2 weeks (for study 2); until behavioral evaluation (study2) at 7 months of age. Mice received a last injection one week before sacrifice (at 8 months of age).

Blood samples were collected at tail vein and plasma were recovered by centrifugation and kept at −20° C. until use in ELISA assays.

Immunohistochemistry (IHC).

Serial free-floating brain coronal sections (40 μm) were obtained using a cryostat (Leica Microsystems GmbH, Germany) kept in a PBS-azide (0.2%) at 4° C. Sections of interest were washed with PBS-Triton (0.2%) and treated for 30 min with 0.3% H2O2, and nonspecific binding was blocked with MOM (Mouse IgG blocking reagent) or goat serum (1/100 in PBS; Vector Laboratories) for 1 hour. The sections were then incubated with the primary antibody (Table 1, below) in PBS-Triton 0.2% overnight at 4° C. After 3 washes (10 min), labeling was amplified using a biotinylated anti-mouse IgG or rabbit-IgG (1/400 in 25 PBS-Triton 0.2%; Vector Laboratories) for 1 hour, followed by the ABC kit (1/400 in PBS; Vector Laboratories), and labeling was completed using 0.5 mg/ml DAB (Vector Laboratories) in 50 mmol/l Tris-HCl, pH 7.6, containing 0.075% H2O2. Brain sections were mounted on SuperFrost slides, dehydrated through a graded series of alcohol and toluene, and then mounted with Vectamount (Vector Laboratories) for microscopic analysis using Zeiss 30 AXIOSCAN Z1 slide scanner and Zen software. Quantifications were performed using ImageJ software; in the same region of interest in the hippocampus.

Biochemical Fractionation (Soluble/Insoluble Tau).

For sarkosyl-soluble/insoluble protein preparations, mice hippocampi were homogenized by sonication in a lysis buffer containing 10 mM Tris-HCl pH7.4, 0.32M sucrose, 800 mM NaCl, 1 mM EGTA with protease inhibitors (Complete, Roche) and centrifuged at 12000 g for 10 minutes at 4° C. Supernatants were incubated in 1% sarkosyl (Sodium N-Lauroyl Sarcosininate, Fluka) under gentle agitation 1 h at room temperature; then centrifuged at 100000 g for 1 h at 4° C. The supernatants containing Sarkosyl soluble Tau species were recovered and the pellets containing insoluble Tau species were directly homogenized in LDS 2λ, supplemented with reducing agents (Invitrogen).

For western blots, protein amounts in soluble fractions were evaluated using the BCA assay (Pierce) and subsequently standardized at 1 μg/u with LDS 2× supplemented with reducing agent (Invitrogen). Sarkosyl-soluble and Sarkosyl-insoluble samples were loaded onto NuPage Novex gels with a ratio of 1:6.

Protein Extractions.

Cells were washed using PBS and harvested in ice-cold RIPA buffer: 150 mM NaCl, 1% NP40, 0.5% sodium deoxycholate, 0.1% SDS, 50 mM Tris-HCl, pH 8.0, completed with protease inhibitors (Complete, Roche). After sonication and homogenization 1 h at 4° C., the supernatants were recovered after centrifugation at 12000 g for 10 minutes at 4° C., and protein concentrations were determined using the BCA Assay Kit (Pierce).

Western Blotting.

For total proteins, extracts were standardized at 1 μg/μl with LDS 2× supplemented with a reducing agent (Invitrogen) and denatured at 100° C. for 10 min. Proteins were then separated with SDS-PAGE using precast 4-12% Bis-Tris NuPage Novex gels (Invitrogen). Proteins were transferred to 0.45 μM nitrocellulose membranes (Amersham™ Hybond ECL), which were saturated with 5% dry non-fat milk in TNT buffer (140 mM NaCl, 0,5% Tween20, 15 mM Tris, pH 7.4), or 5% bovine serum albumin (Sigma) in TNT buffer, according to the primary antibody. Membranes were then incubated with the primary antibody (Table 3, bellow) overnight at 4° C., washed with TNT buffer three times for 10 min, incubated with the secondary antibody (Vector) and washed again. Immunolabeling was visualized using chemiluminescence kits (ECL™, Amersham Bioscience) on LAS-4000 acquisition system (Fujifilm).

Indirect ELISA.

Nunc 96-well microtiter plates (Maxisorp F8; Nunc, Inc.) were coated overnight at 4° C. with 100 ng/well of either Ac-Met11-Tau peptide ({Nα-acetyl}MEDHAGTYGLG), or Tau 1-peptide (MAEPRQEFEVMEDHAGTYGLG) in 50 mM NaHCO₃, pH 9.6. After 3 washes with PBS containing 0.05% Tween (PBS-T), plates were blocked with 0.1% casein solution (PBS) at 37° C. for 1 h, followed by incubation with mice plasma (at 1:600 dilution in PBS containing 0.2% BSA) for 2 h at room temperature. After 3 washes with PBS-T, immunodetection was performed by using a goat anti-mouse IgG horseradish peroxidase-conjugated antibody (A3673; Sigma) at 1:4000 dilution in PBS-BSA 0.2%, at 37° C. for 1 h. After 5 washes with PBS-T, detection was performed using Tetramethyl benzidine substrate (T3405, Sigma) for 30 min at room temperature; the assay was stopped with H2SO4 and absorbance was read with spetrophotometer (Multiskan Ascent, Thermo Labsystems) at 450 nm.

Sandwich ELISA.

Nunc 96-well plates (VWR) were coated with 100 μl of 2H2D11 antibody (for detection of Ac-Met11-Tau species) at a concentration of 1 μg/ml in Carbonate buffer (NaHCO3 0.1M, Na2CO3 0.1M, pH 9.6) overnight at 4° C. The plates were subsequently blocked with a WASH1× buffer (INNOTEST hTau Ag kit, FUJIREBIO) containing 0.1% casein at 37° C. for 1 hour and washed with WASH1× buffer 3 times. Protein samples were standardized at 1 μg/μl and diluted in SAMPL DIL buffer (INNOTEST hTau Ag kit, FUJIREBIO). Protein samples and biotinylated antibodies (HT7/BT2, INNOTEST hTau Ag kit, FUJIREBIO) were added and the plates were incubated at room temperature overnight. The wells were washed four times then incubated with Peroxidase-labeled streptavidin at room temperature for 30 min and washed four times. Detection was performed using Tetramethyl benzidine substrate for 30 min at room temperature; the assay was stopped with H2SO4 and absorbance was read with spetrophotometer (Multiskan Ascent, Thermo Labsystems) at 450 nm. For detection of total Tau proteins or Tau phosphorylated at Ser396, ELISA experiments were performed, accordingly to manufacture's instructions, by using INNOTEST hTau kit (FUJIREBIO) and Human Tau [pS396] ELISA Kit (Invitrogen), respectively.

Y-Maze Studies.

Short-term spatial memory was assessed in a spontaneous novelty-based spatial preference Y-maze test as previously described (Laurent et al., 2016). Each arm of the Y-maze was 22-cm long, 6.4-cm wide, with 15-cm high opaque walls. Different extramaze cues were placed on the surrounding walls. Sawdust was placed in the maze during the experiments and mixed between each phase. Allocation of arms was counterbalanced within each group. During the exposure phase, mice were placed at the end of the ‘start’ arm and were allowed to explore the ‘start’ arm and the ‘other’ arm for 5 min (beginning from the time the mouse first left the start arm). Access to the third arm of the maze (‘novel’ arm) was blocked by an opaque door. The mouse was then removed from the maze and returned to its home cage for 2 min. In the test phase, the mouse was placed again in the ‘start’ arm of the maze, the door of the ‘novel’ arm was removed and the mouse was allowed to explore the maze for 5 min (from the time the mouse first left the start arm). The amount of time the mouse spent in each arm of the maze was recorded during both exposure and test phases using EthovisionXT (Noldus Information Technology).

Statistics.

Images acquisition and quantification as well as behavioral evaluations were performed by investigators blind to the experimental conditions. Results are expressed as means±s.e.m. Differences between mean values were determined using the Student's t-test or one-way ANOVA, followed by a post-hoc Fisher's LSD test using Graphpad Prism Software. P values <0.05 were considered significant.

TABLE 3
Summary of primary antibodies used for Westernbloting, Immunohistochemestry,
Elisa and Immunotherapy.

| Antibody | Epitope | Species | Applicaion (Dilution) | Supplier |
|---|---|---|---|---|
| HT7 | hTau aa159-163 | mouse | WB (1/2000) | Thermofisher #MN1000 |
| Tau-Cter | Tau aa 426-441 | rabbit | WB (1/10000) | Homemade |
| (total Tau) | | | | |
| AT8 | pSer202/pThr205 | mouse | IHC (1/400) | Thermofisher #MN1020 |
| MC1 | Tau aa 312-322 | mouse | IHC (1/1000) | A gift from P. Davies |
| | (conformation dependent) | | | (Gisha et al., 1999) |
| AT100 | pThr212/pSer214 | mouse | WB (1/1000), IHC (1/500) | Invitrogen #MN1060 |
| | (human PHF) | | | |
| 2H2/D11 | AcMet11-Tau | mouse | WB and indirect ELISA | Homemade (WO |
| | | | (1/5000), IHC (1/200), | 201 8/1 78078 Al) |
| | | | Immunotherapy (FIG. 9) | |
| B 69 | Infectious Bursal Disease | mouse | Immunotherapy (FIG. 9) | Home-purified from: B69 |
| | (IBD) virus | | | ATCC HB-9437 |
| | | | | hybridoma |
| GAPDH | Mouse GAPDH FL1-335 | rabbit | WB (1/10000) | Sigma-Aldrich #G9545 |

Results

Our earliest data have shown AcMet11-Tau to be a signature feature of AD related-Tau pathology (WO 2018/178078).

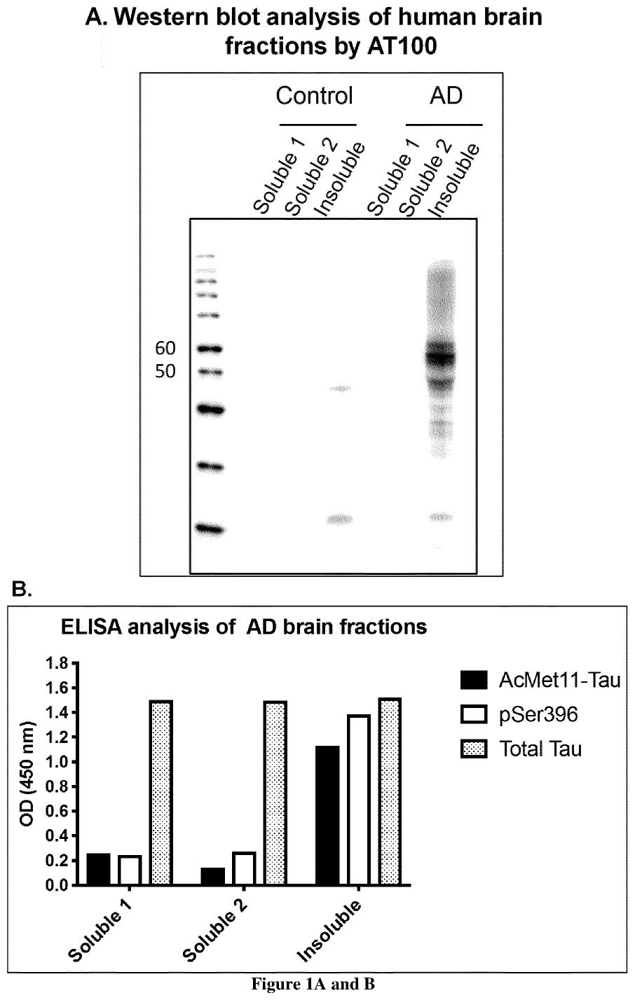

We have performed further analyses and data indicate that AcMet11-Tau are pathological Tau species that are involved in Tau pathology development. First, we have done biochemical fractionation of AD brain proteins. Our ELISA analyses showed that like pathological hyperphosphorylated Tau proteins, AcMet11-Tau species are present in the insoluble fraction (FIG. 1)

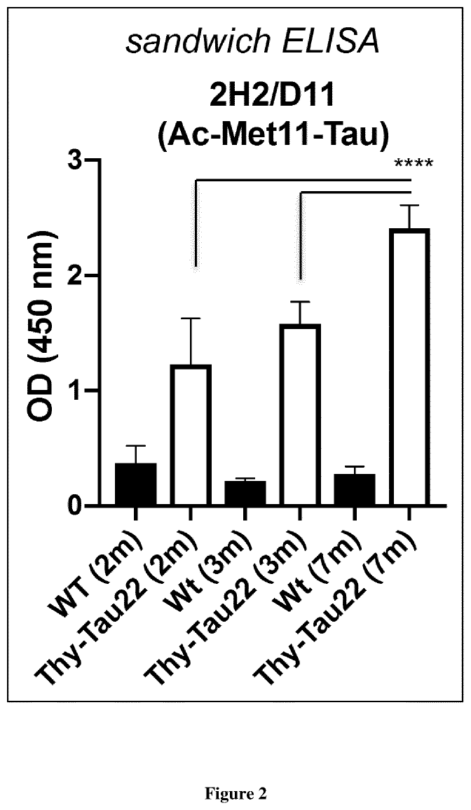

Second, in THY-Tau22 mice that gradually develop hippocampal Tau pathology from 3 to 10 months and memory deficits from 6 months (Schindowski et al., 2006; Van der Jeugd at al., 2013), AcMet11-Tau species are detected at early stages of the pathological process that precede memory deficits. Indeed, immunohistochemical analyses of hippocampal brain sections (not shown) and Sandwich ELISA using hippocampal protein extracts (FIG. 2) have shown AcMet11-Tau to be detected in Thy-Tau22 mice at 2 months of age, while memory deficits are detected from 6 months.

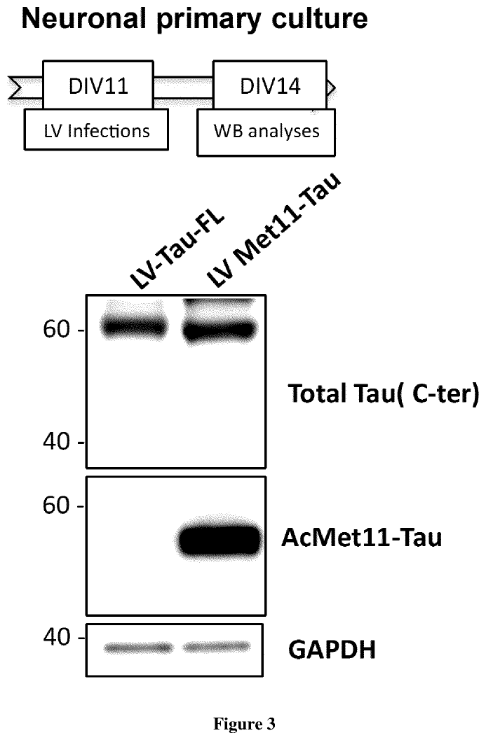

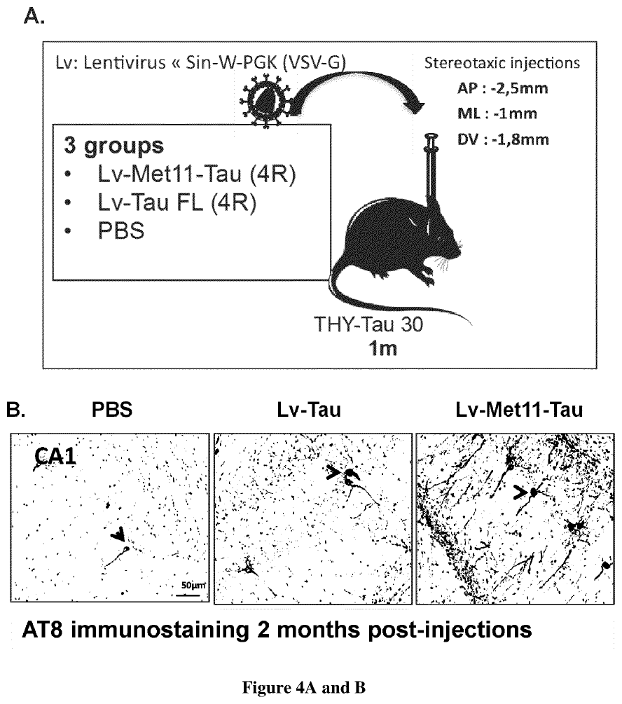

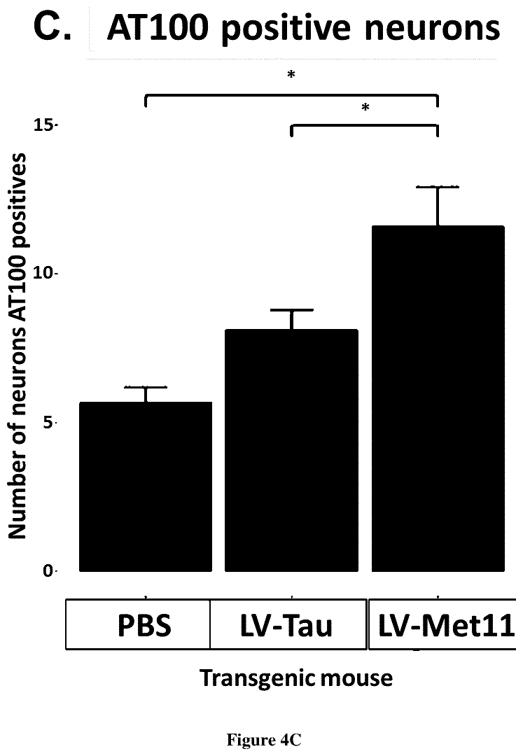

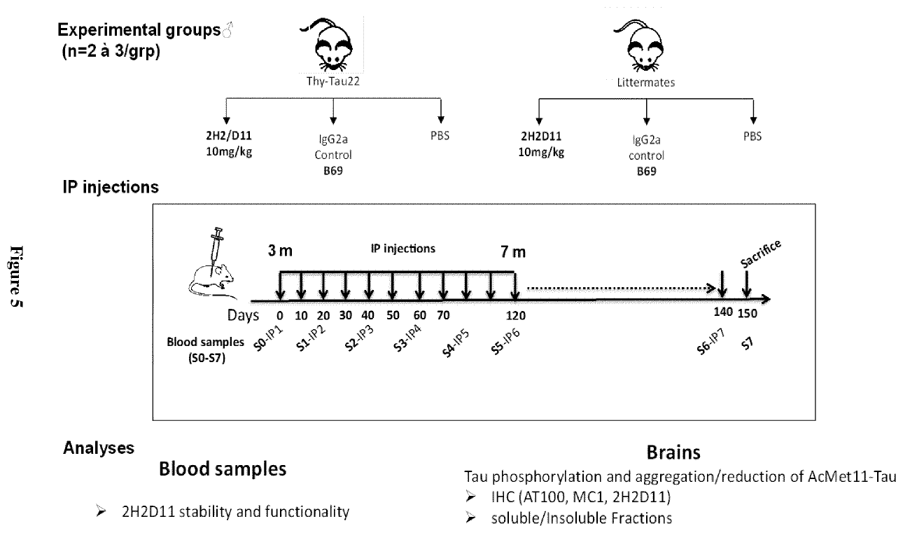

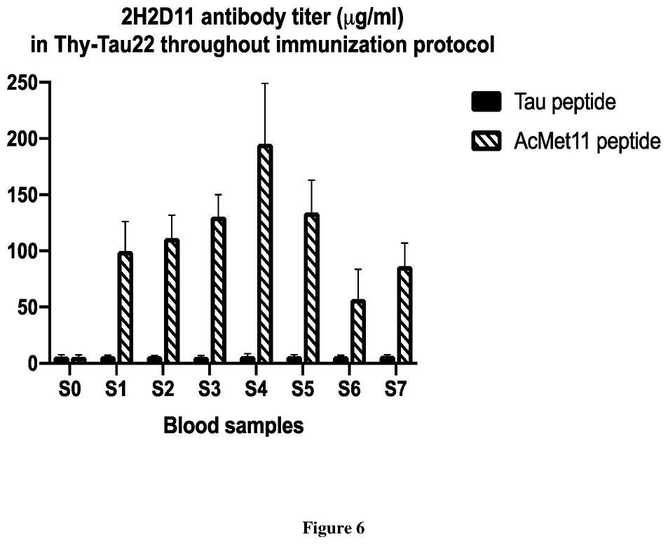

Third, We have evaluated the causal link between AcMet11-Tau species and Tau pathology by analyzing whether brain expression of AcMet11-Tau species potentiate Tau pathology development in Thy-Tau30 Transgenic mice. We have performed stereotaxic hippocampal injections of lentiviral vectors (LV) allowing neuronal expression (Caillierez et al., 2013) of either Met11-Tau or full-length Tau protein (FL-Tau). Noteworthy, we have ensured prior to stereotaxic injections that LV batches allow expression of FL-Tau and Met11-Tau at the same level in primary neuronal cells and that Met11-Tau is expressed as N-α-acetylated form (FIG. 3). Our IHC analyses of brain sections of mice, performed 2 months post-stereotaxic injections of LV, showed that Tau proteins are stably expressed in neuronal cells within the hippocampus and that Met11-Tau is detected as N-α-acetylated form, especially in regions that are relevant to synaptic plasticity and memory, i.e. CA1, CA3, Dendrite Gyrus (DG) and mossy fibers (not shown). Importantly, immunostaining, using AT8 and AT100 antibodies, indicated that mice expressing AcMet11-Tau displayed an increased number of neurofibrillary tangles-bearing neurons, compared to mice expressing FL-Tau (FIG. 4), suggesting hence that AcMet11-Tau species are involved in pathological process, at least by accelerating Tau pathology.

Overall, our data indicate that AcMet11-Tau is an early pathological species of physiopathological value and could hence be a valuable therapeutic target. Then, we have evaluated whether reduction/neutralization of this Tau species in the Thy-Tau22 transgenic model of Tau pathology lead to protective effect towards Tau pathology and associated memory deficits. To achieve this aim, we have used passive immunization approach based on the specific monoclonal antibody (2H2D11) we have developed against AcMet11-Tau species.

We have first performed a pilot study with a limited number of mice (n=3/group) to evaluate the experimental setup of 2H2D11-based immunotherapy.

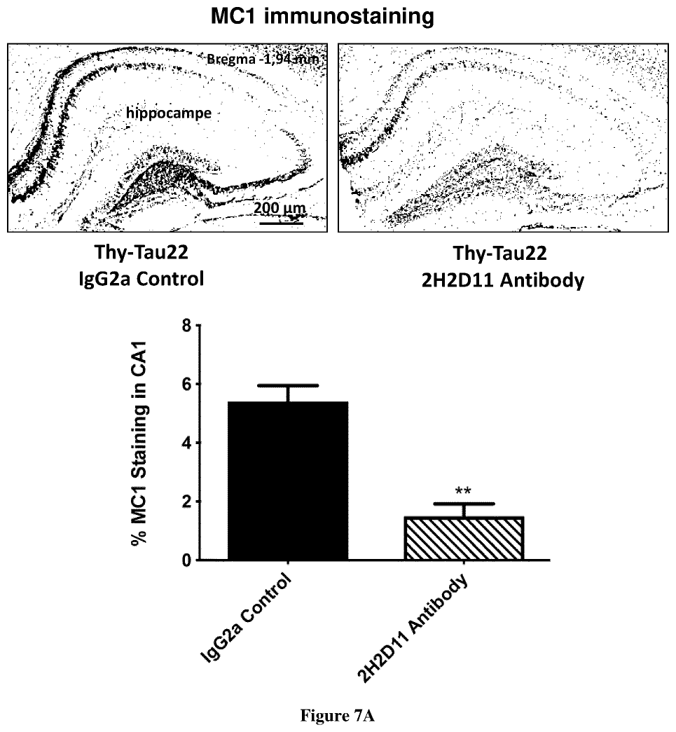

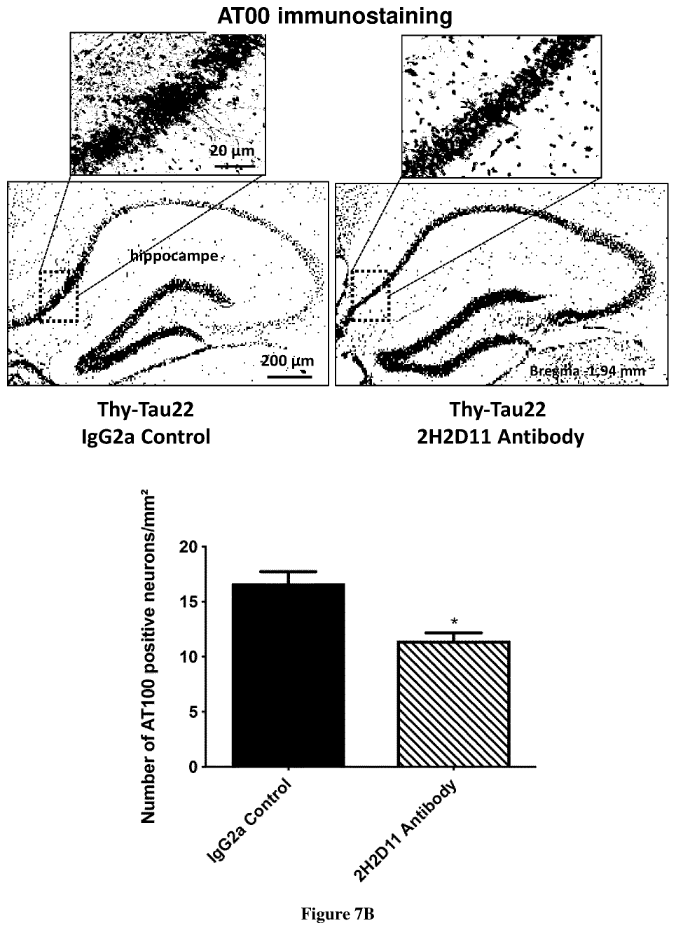

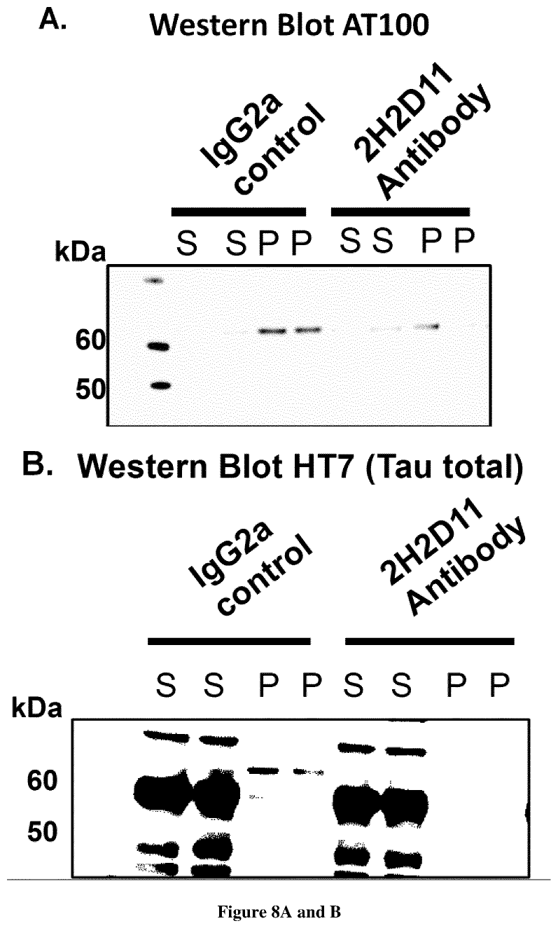

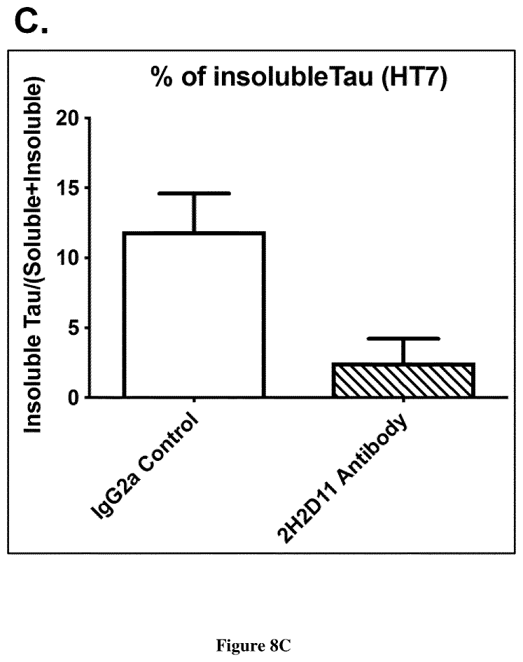

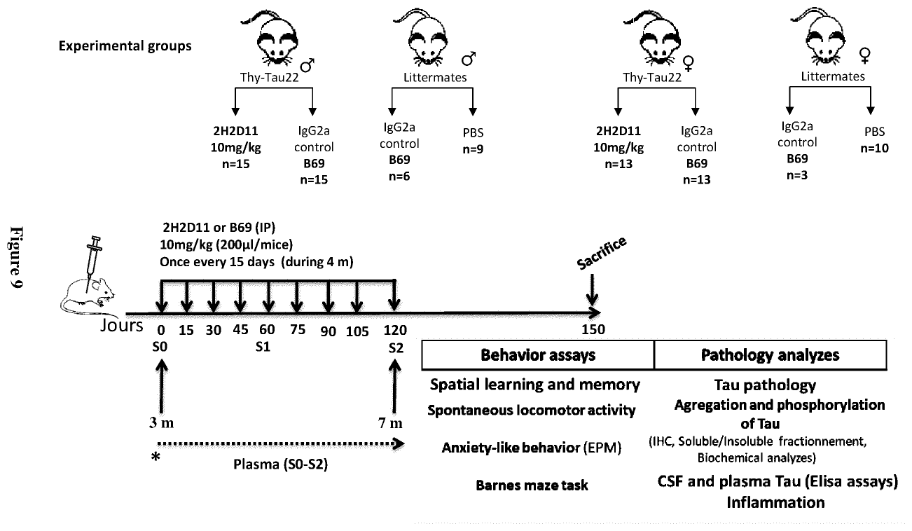

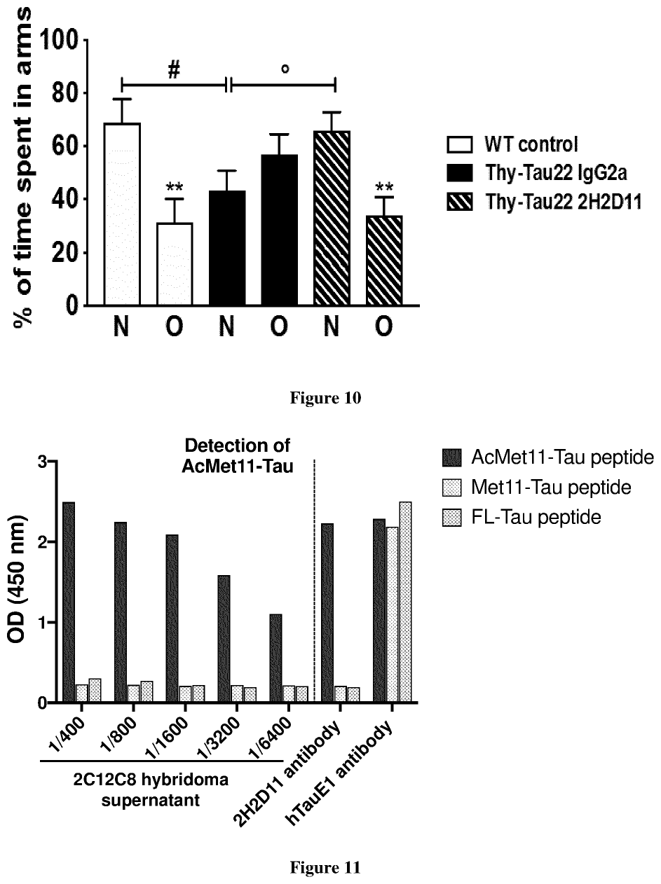

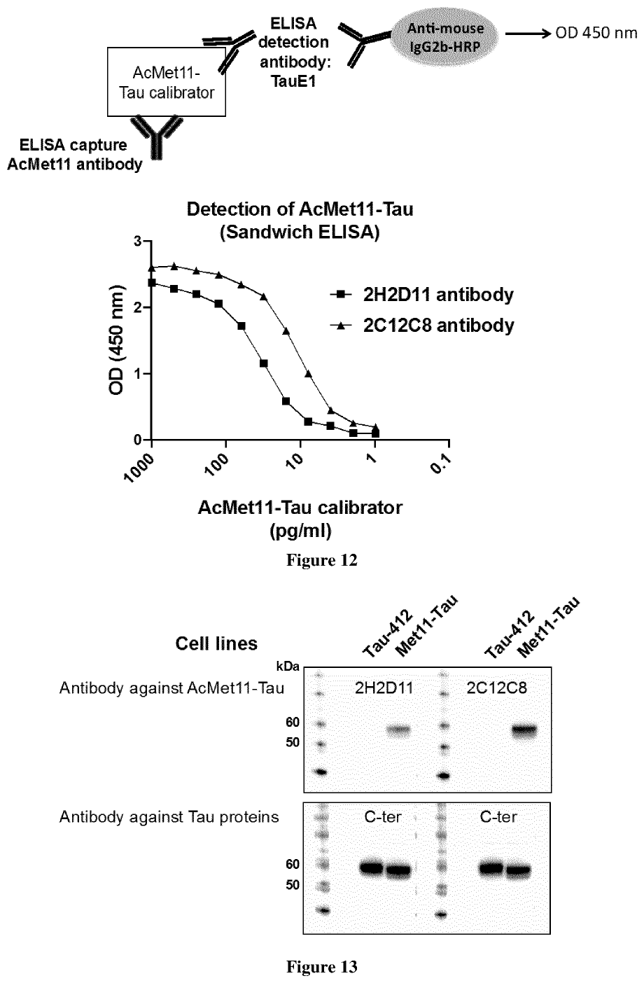

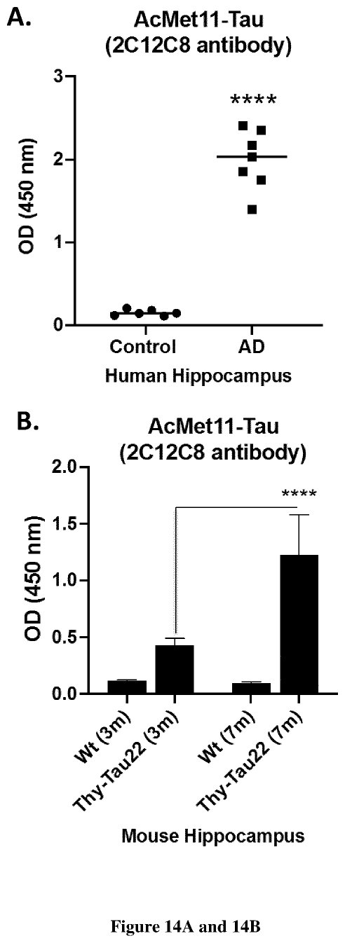

Thy-Tau22 and their littermate WT mice have been injected every 10 days (intraperitoneal (IP) injections), from 3 months (early pathological stage in Thy-Tau22 mice) to 7-months of age when Tau pathology and memory impairment are present but not maximal in this model. The repeated IP injections were performed with either PBS or 10 mg/kg of the monoclonal antibody directed against AcMet11-Tau (2H2D11 antibody; IgG2a isotype) as well as with a non-specific antibody (IgG2a isotype control antibody) (FIG. 5). Our analyses showed no overt toxicity related to immunotherapy and found the 2H2D11 antibody is stable and functional in blood over at least 20 days (FIG. 6). Importantly, we obtained exciting data from Tau pathology analyses. Indeed, IHC staining using antibodies against specific conformational and pathological Tau epitopes, MC1 (FIG. 7A) and AT100 (FIG. 7B), respectively, obviously showed a significant decrease in Tau pathology in the hippocampus of Thy-Tau22 mice injected with 2H2D11 antibody. Likewise, analyses of insoluble fractions from hippocampal proteins showed that 2H2D11-based immunotherapy induced a decrease in pathological Tau insoluble species (FIG. 8).

Thereafter, we have undertaken a broad passive immunotherapy study with a large number of mice (FIG. 9). Following 4 months of treatments, the impact of immunotherapy was evaluated on short-term spatial memory. Data showed that compared to WT mice, and as expected for Thy-Tau22 line (Laurent et al., 2016), Thy-Tau22 mice injected with IgG2a control exhibited impaired spatial memory as demonstrated by the lack of preference for the novel arm (FIG. 10). In contrast, Thy-Tau22 mice treated with 2H2D11 antibody performed like WT mice, spending significantly more time in the novel arm than the other arm, revealing hence the beneficial effect of anti AcMet11-Tau immunotherapy towards spatial memory.

### CONCLUSION

We have established the proof-of-concept that AcMet11-Tau species are a good therapeutic targets in the field of Tau pathology related to AD and Tauopathies, especially as a target for immunotherapy. Our previous data have clearly showed AcMet11-Tau to be exclusively detected in the pathological context (WO 2018/178078). Here we showed that AcMet11-11 have a driving role in Tau pathology development. Importantly, passive immunotherapy, using unique antibodies we have developed against AcMet11-Tau species, showed a beneficial impact by reducing Tau pathology and by improving memory deficits in a transgenic model of Tau pathology.

### EXAMPLE 2

In order to get further antibodies against N-alpha-acetyl-Met11-Tau (AcMet11-Tau), we subcloned 2C12 hybridoma (described in in Table 2 of Example 1) and we have selected the 2C12C8 clone. Isotype strips showed that 2C12C8 hybridoma produced an IgG1 antibody with kappa chain while we have previously established (see in Table 2 of Example 1) that 2H2D11 is an IgG2a antibody. As shown in FIG. 11, indirect ELISA was used to analyze different dilutions of culture supernatant from 2C12C8 hybridoma. Data indicated that similarly to 2H2D11 antibody (used as positive control), supernatant from 2C12C8 hybridoma displayed specificity towards N-alpha-terminally acetylated methionine11 of Tau protein. Indeed, 2C12C8 displays no reactivity with the free non-N-alpha-terminally-acetylated Methionine 11 (Met11-Tau peptide), or with the non-truncated Methionine 11 (Tau1 peptide), or with an N-alpha-acetyl-Methionine when it is not in the same amino acid context than Methionine11 (FL-Tau peptide). Regarding 7C12/E7 antibody against total Tau proteins (hTauE1 antibody), it displays the similar immunoreactivity towards the 3 different peptides.

After purification of 2C12C8 antibody (accordingly to the protocol described in WO 2018/178078), this monoclonal antibody was further characterized by sandwich ELISA using serial dilutions of an AcMet11-Tau calibrator. Our data (FIG. 12) showed that similarly to 2H2D11 antibody, 2C12C8 antibody is also able to detect AcMet11-Tau by sandwich ELISA.

The specificity of 2C12C8 antibody was validated by Western Blotting (FIG. 13) using protein extracts from the cell lines overexpressing either AcMet11-Tau (Met11-Tau) or full-length Tau (Tau-412) (described in WO 2018/178078). Expression of full-length-Tau and truncated Tau is displayed by an antibody against C-terminal part of Tau in Tau-412 and Met11-Tau cells, respectively. However, analysis using either 2H2D11 antibody or 2C12C8 antibody displayed immunolabelling only with extracts from Met11-Tau cells.

After 2C12C8 characterisation, we analysed whether similarly to 2H2D11 antibody (FIG. 2 and WO 2018/178078), 2C12C8 antibody was also able to show an association between AcMet11-Tau species and Tau pathology. Protein extracts from hippocampus of elderly controls (n=6) and AD patients (n=7; from Braak IV-VI; described in WO 2018/178078) were analysed by 2C12C8-based sandwich ELISA (FIG. 14A). As described previously by using 2H2D11 antibody (WO 2018/178078), 2C12C8 antibody specifically reacted with AD hippocampus samples (p<0.0001; compared with unpaired t test). Likewise, 2C12C8 antibody allowed specific detection of AcMet11-Tau in Thy-Tau22 transgenic mice by sandwich ELISA (FIG. 14B). The immunoreactivity in Thy-Tau22 transgenic mice displayed a significant increase with age (7 months versus 3 months; p=0.0016; compared with unpaired t test; n=4/group). Moreover, immunohistochemistry analysis of hippocampus sections from these mice showed that like 2H2D11 antibody (WO 2018/178078), 2C12C8 displayed no immunoreactivity with Wt mice (FIG. 15, panel A) while in Thy-Tau22 mice 2C12C8 antibody labels Tau pathology as displayed by typical pathological inclusions in neurons (FIG. 15, panels B-D). As showed previously with 2H2D11 antibody (WO 2018/178078), 2C12C8 antibody immunolabelling is detected early during pathological process (FIG. 15, panel B).

Finally, passive immunization approach based on the specific monoclonal antibody (2C12C8) developed against AcMet11-Tau species, as 2H2D11 antibody, is also under investigation in a transgenic model of Tau pathology.

TABLE 4
Useful (amino acid and nucleic) sequences for practicing the invention

| SEQ ID NO | Nucleotide or amino acid sequence |
|---|---|
| 1: Tau N-Alpha | N-Alpha Acetyl-MEDHAGTYGL GDRKDQGGYT MHQDQEGDTD |
| Acetyl-Met11- | AGLKAEEAGI GDTPSLEDEA AGHVTQARMV SKSKDGTGSD |
| 352 | DKKAKGADGK TKIATPRGAA PPGQKGQANA TRIPAKTPPA |
| | PKTPPSSGEP PKSGDRSGYS SPGSPGTPGS RSRTPSLPTP |
| | PTREPKKVAV VRTPPKSPSS AKSRLQTAPV PMPDLKNVKS |
| | KIGSTENLKH QPGGGKVQIV YKPVDLSKVT SKCGSLGNIH |
| | HKPGGGQVEV KSEKLDFKDR VQSKIGSLDN ITHVPGGGNK |
| | KIETHKLTFR ENAKAKTDHG AEIVYKSPVV SGDTSPRHLS |
| | NVSSTGSIDM VDSPQLATLA DEVSASLAKQ GL |
| 2: Tau N-Alpha | N-Alpha Acetyl MEDHAGTYGL GDRKDQGGYT MHQDQEGDTD |
| Acetyl-Met11- | AGLKESPLQT PTEDGSEEPG SETSDAKSTP TAEAEEAGIG |
| 381 | DTPSLEDEAA GHVTQARMVS KSKDGTGSDD KKAKGADGKT |
| | KIATPRGAAP PGQKGQANAT RIPAKTPPAP KTPPSSGEPP |
| | KSGDRSGYSS PGSPGTPGSR SRTPSLPTPP TREPKKVAVV |
| | RTPPKSPSSA KSRLQTAPVP MPDLKNVKSK IGSTENLKHQ |
| | PGGGKVQIVY KPVDLSKVTS KCGSLGNIHH KPGGGQVEVK |
| | SEKLDFKDRV QSKIGSLDNI THVPGGGNKK IETHKLTFRE |
| | NAKAKTDHGA EIVYKSPVVS GDTSPRHLSN VSSTGSIDMV |
| | DSPQLATLAD EVSASLAKQG L |
| 3: Tau N-Alpha | N-Alpha Acetyl MEDHAGTYGL GDRKDQGGYT MHQDQEGDTD |
| Acetyl-Met11- | AGLKAEEAGI GDTPSLEDEA AGHVTQARMV SKSKDGTGSD |
| 383 | DKKAKGADGK TKIATPRGAA PPGQKGQANA TRIPAKTPPA |
| | PKTPPSSGEP PKSGDRSGYS SPGSPGTPGS RSRTPSLPTP |
| | PTREPKKVAV VRTPPKSPSS AKSRLQTAPV PMPDLKNVKS |
| | KIGSTENLKH QPGGGKVQII NKKLDLSNVQ SKCGSKDNIK |
| | HVPGGGSVQI VYKPVDLSKV TSKCGSLGNI HHKPGGGQVE |
| | VKSEKLDFKD RVQSKIGSLD NITHVPGGGN KKIETHKLTF |
| | RENAKAKTDH GAEIVYKSPV VSGDTSPRHL SNVSSTGSID |
| | MVDSPQLATL ADEVSASLAK QGL |
| 4. Tau N-Alpha | N-Alpha Acetyl MEDHAGTYGL GDRKDQGGYT MHQDQEGDTD |
| Acetyl-Met11- | AGLKESPLQT PTEDGSEEPG SETSDAKSTP TAEDVTAPLV |
| 410 | DEGAPGKQAA AQPHTEIPEG TTAEEAGIGD TPSLEDEAAG |
| | HVTQARMVSK SKDGTGSDDK KAKGADGKTK IATPRGAAPP |
| | GQKGQANATR IPAKTPPAPK TPPSSGEPPK SGDRSGYSSP |
| | GSPGTPGSRS RTPSLPTPPT REPKKVAVVR TPPKSPSSAK |
| | SRLQTAPVPM PDLKNVKSKI GSTENLKHQP GGGKVQIVYK |
| | PVDLSKVTSK CGSLGNIHHK PGGGQVEVKS EKLDFKDRVQ |
| | SKIGSLDNIT HVPGGGNKKI ETHKLTFREN AKAKTDHGAE |
| | IVYKSPVVSG DTSPRHLSNV SSTGSIDMVD SPQLATLADE |
| | VSASLAKQGL |
| 5. Tau N-Alpha | N-Alpha Acetyl-MEDHAGTYGL GDRKDQGGYT MHQDQEGDTD |
| Acetyl-Met11- | AGLKESPLQT PTEDGSEEPG SETSDAKSTP TAEAEEAGIG |
| 412 | DTPSLEDEAA GHVTQARMVS KSKDGTGSDD KKAKGADGKT |
| | KIATPRGAAP PGQKGQANAT RIPAKTPPAP KTPPSSGEPP |
| | KSGDRSGYSS PGSPGTPGSR SRTPSLPTPP TREPKKVAVV |
| | RTPPKSPSSA KSRLQTAPVP MPDLKNVKSK IGSTENLKHQ |
| | PGGGKVQIIN KKLDLSNVQS KCGSKDNIKH VPGGGSVQIV |
| | YKPVDLSKVT SKCGSLGNIH HKPGGGQVEV KSEKLDFKDR |
| | VQSKIGSLDN ITHVPGGGNK KIETHKLTFR ENAKAKTDHG |
| | AEIVYKSPVV SGDTSPRHLS NVSSTGSIDM VDSPQLATLA |
| | DEVSASLAKQ GL |
| 6 : Tau N-Alpha | N-Alpha Acetyl-MEDHAGTYGL GDRKDQGGYT MHQDQEGDTD |
| Acetyl-Met11- | AGLKESPLQT PTEDGSEEPG SETSDAKSTP TAEDVTAPLV |
| 441 (Isoform2) | DEGAPGKQAA AQPHTEIPEG TTAEEAGIGD TPSLEDEAAG |
| | HVTQARMVSK SKDGTGSDDK KAKGADGKTK IATPRGAAPP |
| | GQKGQANATR IPAKTPPAPK TPPSSGEPPK SGDRSGYSSP |
| | GSPGTPGSRS RTPSLPTPPT REPKKVAVVR TPPKSPSSAK |
| | SRLQTAPVPM PDLKNVKSKI GSTENLKHQP GGGKVQIINK |
| | KLDLSNVQSK CGSKDNIKHV PGGGSVQIVY KPVDLSKVTS |
| | KCGSLGNIHH KPGGGQVEVK SEKLDFKDRV QSKIGSLDNI |
| | THVPGGGNKK IETHKLTFRE NAKAKTDHGA EIVYKSPVVS |
| | GDTSPRHLSN VSSTGSIDMV DSPQLATLAD EVSASLAKQG L |
| 7. Tau N-Alpha | N-Alpha Acetyl-MEDHAGTYGL GDRKDQGGYT MHQDQEGDTD |
| Acetyl-Met11- | AGLKESPLQT PTEDGSEEPG SETSDAKSTP TAEDVTAPLV |
| 776 | DEGAPGKQAA AQPHTEIPEG TTAEEAGIGD TPSLEDEAAG |
| | HVTQEPESGK VVQEGFLREP GPPGLSHQLM SGMPGAPLLP |
| | EGPREATRQP SGTGPEDTEG GRHAPELLKH QLLGDLHQEG |
| | PPLKGAGGKE RPGSKEEVDE DRDVDESSPQ DSPPSKASPA |
| | QDGRPPQTAA REATSIPGFP AEGAIPLPVD FLSKVSTEIP |
| | ASEPDGPSVG RAKGQDAPLE FTFHVEITPN VQKEQAHSEE |
| | HLGRAAFPGA PGEGPEARGP SLGEDTKEAD LPEPSEKQPA |
| | AAPRGKPVSR VPQLKARMVS KSKDGTGSDD KKAKTSTRSS |
| | AKTLKNRPCL SPKHPTPGSS DPLIQPSSPA VCPEPPSSPK |
| | YVSSVTSRTG SSGAKEMKLK GADGKTKIAT PRGAAPPGQK |
| | GQANATRIPA KTPPAPKTPP SSATKQVQRR PPPAGPRSER |
| | GEPPKSGDRS GYSSPGSPGT PGSRSRTPSL PTPPTREPKK |
| | VAVVRTPPKS PSSAKSRLQT APVPMPDLKN VKSKIGSTEN |
| | LKHQPGGGKV QIINKKLDLS NVQSKCGSKD NIKHVPGGGS |
| | VQIVYKPVDL SKVTSKCGSL GNIHHKPGGG QVEVKSEKLD |
| | FKDRVQSKIG SLDNITHVPG GGNKKIETHK LTFRENAKAK |
| | TDHGAEIVYK SPVVSGDTSP RHLSNVSSTG SIDMVDSPQL |
| | ATLADEVSAS LAKQGL |
| 8. Tau N-Alpha | N-Alpha Acetyl-MEDHAGTYGLG |
| Acetyl-Met11- | |
| 21 (epitope and | |
| antigen) | |
| 9. Tau Met11-21 | MEDHAGTYGLG |
| 10. Tau N-Alpha | N-Alpha Acetyl-MAEPRQEFEV MEDHAGTYGLG |
| Acetyl-Met1-21 | |
| 11 H-CDR1 of | DYYLS |
| 2H2D11 | |
| 12. H-CDR2 of | NRADGYTTNYSASVKG |
| 2H2D11 | |
| 13. H-CDR3 of | DNDHYAMDY |
| 2H2D 11 | |
| 14. L-CDR1 of | KSSQSLLYSNGKTYLN |
| 2H2D11 | |
| 15. L-CDR2 of | QVSKLDP |
| 2H2D11 | |
| 16. L-CDR3 of | LQGTYYPYT |
| 2H2D11 | |
| 17. VH-of | EVRLVESGGGLVQPGGSLRLSCATSGFTFTDYYLSWVRQPPG |
| 2H2D11 | KAFEWLGFIRNRADGYTTNYSASVKGRFTISRDNSQSILYLQM |
| | NTLRVEDSATYYCARDNDHYAMDYWGQGTSVTVSS |
| 18. VL-of | DVVMTQTPLSLSVTIGQPASISCKSSQSLLYSNGKTYLNWLQQ |
| 2H2D11 | RPGQAPKHLMYQVSKLDPGIPDRFSGSGSETDFILKISRVEAED |
| | LGIYYCLQGTYYPYTFGGGTKLEIK |
| 19. VH of | gaggtgaggctggtggagtctggaggaggcttggtacagcctgggggttctctgagactctcctgt |
| 2H2D11 | gcaacttctgggttcaccttcactgattactacctgagctgggtccgccagcctccaggaaaggcat |
| (nucleic acid | ttgagtggttgggattattagaaacagagctgatggttacacaacaaactacagtgcatctgtgaag |
| sequence) | ggtcggttcaccatctccagagataattcccaaagcatcctctatcttcaaatgaacaccctgagagt |
| | tgaggacagtgccacttattactgtgcaagagacaatgatcactatgctatggactactggggtcaa |
| | ggaacctcagtcaccgtctcttca |
| 20. VL of | gatgttgtgatgacccagactccactgtattgteggttaccattggacaaccagcctctatctcttgc |
| 2H2D11 | aagtcgagtcagagcctcttatatagtaatggaaagacatatttgaattggttacaacagaggcctg |
| (nucleic acid | gccaggctccaaagcacctaatgtatcaggtgtccaaactggaccctggcatccctgacaggttca |
| sequence) | gtggcagtggatcagaaacagattttatacttaaaattagcagagtggaggctgaagatttgggaat |
| | ttattactgatgcaaggtacatattatccgtacacgttcggaggggggaccaagttggaaataaaa |

### REFERENCES

Throughout this application, various references describe the state of the art to which this invention pertains. The disclosures of these references are hereby incorporated by reference into the present disclosure.
Basurto-Islas G, Luna-Muñoz J, Guillozet-Bongaarts A L, Binder L I, Mena R, Garcia-Sierra F. Accumulation of aspartic acid421- and glutamic acid391-cleaved tau in neurofibrillary tangles correlates with progression in Alzheimer disease. J Neuropathol Exp Neurol. 2008 May; 67(5):470-83.
Braak H., Braak E. Staging of Alzheimer's disease-related neurofibrillary changes. Neurobiol Aging. 1995; 16:271-8; discussion 278-84
Braak, H., Thal, D. R., Ghebremedhin, E. & Del Tredici, K. Stages of the pathologic process in Alzheimer disease: age categories from 1 to 100 years. J Neuropathol Exp Neurol 70, 960-969 (2011).
Caillet-Boudin M L, Buée L, Sergeant N, Lefebvre B. Regulation of human MAPT gene expression. Mol Neurodegener. 14; 10:28 (2015).
Caillierez R, Bégard S, Lécolle K, Deramecourt V, Zommer N, Dujardin S, Loyens A, Dufour N, Aurégan G, Winderickx J, Hantraye P, Déglon N, Buée L, Colin M. Lentiviral delivery of the human wild-type tau protein mediates a slow and progressive neurodegenerative tau pathology in the rat brain. Mol Ther. 2013 July; 21(7):1358-68.
Carmel G, Mager E M, Binder L I, Kuret J. The structural basis of monoclonal antibody Alz50's selectivity for Alzheimer's disease pathology. J Biol Chem. 271:32789-95 (1996).
Déglon, N., J. L. Tseng, J. C. Bensadoun, A. D. Zurn, Y. Arsenijevic, L. Pereira de Almeida, R. Zufferey, D. Trono, and P. Aebischer. 2000. “Self-Inactivating Lentiviral Vectors with Enhanced Transgene Expression as Potential Gene Transfer System in Parkinson's Disease.” Human Gene Therapy 11 (1): 179-90.
Delacourte A, David J P, Sergeant N, Buée L, Wattez A, Vermersch P, Ghozali F, Fallet-Bianco C, Pasquier F, Lebert F, Petit H, Di Menza C. The biochemical pathway of neurofibrillary degeneration in aging and Alzheimer's disease. Neurology 52, 1158-1165 (1999).
Derisbourg M, Leghay C, Chiappetta G, Fernandez-Gomez F J, Laurent C, Demeyer D, Carrier S, Buée-Scherrer V, Blum D, Vinh J, Sergeant N, Verdier Y, Buée L, Hamdane M. Role of the Tau N-terminal region in microtubule stabilization revealed by new endogenous truncated forms. Sci Rep. 5:9659 (2015).
Duyckaerts C, Colle M A, Dessi F, Piette F, Hauw J J. Progression of Alzheimer histopathological changes. Acta Neurol Belg. 1998; 98:180-5.
Garcia-Sierra F, Wischik C M, Harrington C R, Luna-Muñoz J, Mena R. Accumulation of C-terminally truncated tau protein associated with vulnerability of the perforant pathway in early stages of neurofibrillary pathology in Alzheimer's disease. J Chem Neuroanat. 2001 July; 22(1-2):65-77.
Hayashi S, Toyoshima Y, Hasegawa M, Umeda Y, Wakabayashi K, Tokiguchi S, Iwatsubo T, Takahashi H. Late-onset frontotemporal dementia with a novel exon 1 (Arg5His) tau gene mutation. Ann. Neurol. 51, 525-530 (2002).
Ittner A, Bertz J, Suh L S, Stevens C H, Gotz J, Ittner L M. Tau-targeting passive immunization modulates aspects of pathology in tau transgenic mice. J Neurochem. 2015 January; 132(1):135-45.
Jeganathan S, von Bergen M, Brutlach H, Steinhoff H J, Mandelkow E. Global hairpin folding of tau in solution. Biochemistry. 45:2283-93 (2006).
Jeganathan S, Hascher A, Chinnathambi S, Biernat J, Mandelkow E M, Mandelkow E. Proline-directed pseudo-phosphorylation at AT8 and PHF1 epitopes induces a compaction of the paperclip folding of Tau and generates a pathological (MC-1) conformation. J Biol Chem. 283:32066-76 (2008).
Jicha G A, Berenfeld B, Davies P. Sequence requirements for formation of conformational variants of tau similar to those found in Alzheimer's disease. J Neurosci Res. 1999 Mar. 15; 55(6):713-23.
Laurent C, Burnouf S, Ferry B, Batalha V L, Coelho J E, Baqi Y, Malik E, Mariciniak E, Parrot S, Van der Jeugd A, Faivre E, Flaten V, Ledent C, D'Hooge R, Sergeant N, Hamdane M, Humez S, Müller C E, Lopes L V, Buée L, Blum D. A2A adenosine receptor deletion is protective in a mouse model of Tauopathy. Mol Psychiatry. 2016 January; 21(1):97-107.
Laurent C, Dorothée G, Hunot S, Martin E, Monnet Y, Duchamp M, Dong Y, Légeron F P, Leboucher A, Burnouf S, Faivre E, Carvalho K, Caillierez R, Zommer N, Demeyer D, Jouy N, Sazdovitch V, Schraen-Maschke S, Delarasse C, Buée L, Blum D. Hippocampal T cell infiltration promotes neuroinflammation and cognitive decline in a mouse model of tauopathy. Brain. 2017 January; 140(1):184-200.
Leroy K, Bretteville A, Schindowski K, Gilissen E, Authelet M, De Decker R, Yilmaz Z, Buée L, Brion J P. Early axonopathy preceding neurofibrillary tangles in mutant tau transgenic mice. Am J Pathol. 2007 September; 171(3):976-92.
Magnani E, Fan J, Gasparini L, Golding M, Williams M, Schiavo G, Goedert M, Amos L A, Spillantini M G. Interaction of tau protein with the dynactin complex. EMBO J. 26:4546-54 (2007).
McMillan P J, Kraemer B C, Robinson L, Leverenz J B, Raskind M, Schellenberg G. Truncation of tau at E391 promotes early pathologic changes in transgenic mice. J Neuropathol Exp Neurol. 2011 November; 70(11):1006-19.
Novak P, Kontsekova E, Zilka N, Novak M. Ten Years of Tau-Targeted Immunotherapy: The Path Walked and the Roads Ahead. Front Neurosci. 2018 Nov. 2; 12:798.
Pedersen J T, Sigurdsson E M. Tau immunotherapy for Alzheimer's disease. Trends Mol Med. 2015 June; 21(6):394-402.
Poorkaj P, Muma N A, Zhukareva V, Cochran E J, Shannon K M, Hurtig H, Koller W C, Bird T D, Trojanowski J Q, Lee V M, Schellenberg G D. An R5L tau mutation in a subject with a progressive supranuclear palsy phenotype. Ann. Neurol. 52, 511-516 (2002).
Rissman R A, Poon W W, Blurton-Jones M, Oddo S, Torp R, Vitek M P, LaFerla F M, Rohn T T, Cotman C W. Caspase-cleavage of tau is an early event in Alzheimer disease tangle pathology. J Clin Invest. 2004 July; 114(1):121-30.
Sigurdsson E M. Tau Immunotherapy. Neurodegener Dis. 2016; 16(1-2):34-8.
Schindowski K, Bretteville A, Leroy K, Bégard S, Brion J P, Hamdane M, Buée L. Alzheimer's disease-like tau neuropathology leads to memory deficits and loss of functional synapses in a novel mutated tau transgenic mouse without any motor deficits. Am J Pathol. 169:599-616. (2006).
Van der Jeugd A, Blum D, Raison S, Eddarkaoui S, Buée L, D'Hooge R. Observations in THY-Tau22 mice that resemble behavioral and psychological signs and symptoms of dementia. Behav Brain Res. 242:34-9 (2013).
Wisniewski T, Goñi F. Immunotherapeutic approaches for Alzheimer's disease. Neuron. 2015 Mar. 18; 85(6):1162-76.
WO 2018/178078: New Tau species. Buée L, Hamdane M, Blum D, Derisbourg M, Leghay C, Chiappetta G, Vinh J, Verdier Y. Mar. 27, 2018.
Zilka N, et al. The self-perpetuating tau truncation circle. Biochem Soc Trans 40, 681-686 (2012). Braak H, Braak E. Staging of Alzheimer's disease-related neurofibrillary changes. Neurobiol Aging. 1995; 16:271-8; discussion 278-84
Zilka N, et al. Truncated tau from sporadic Alzheimer's disease suffices to drive neurofibrillary degeneration in vivo. FEBS Lett. 2006 Jun. 26; 580(15):3582-8.

1. A method of treating a Tauopathy disorder in a subject in need thereof, comprising administering to the subject a therapeutically effective amount of an anti-Tau antibody, wherein said anti-Tau antibody binds to an epitope comprising the following amino acid sequence: (N-α-acetyl)MEDHAGTYGLG (SEQ ID NO:8).

2. The method according to claim 1 wherein the anti-Tau antibody specifically binds a Tau polypeptide starting from the methionine residue at position 11, wherein said methionine residue at position 11 is N-alpha acetylated (Ac11Met-Tau).

3. The method according to claim 2 wherein the anti-Tau antibody specifically binds a Tau polypeptide selected from the group consisting of:
(i) the amino acid sequence Tau N-Alpha Acetyl-Met11-352 (SEQ ID NO:1);
(ii) the amino acid sequence Tau N-Alpha Acetyl-Met11-381 (SEQ ID NO:2);
(iii) the amino acid sequence Tau N-Alpha Acetyl-Met11-383 (SEQ ID NO:3)
(iv) the amino acid sequence Tau N-Alpha Acetyl-Met11-410 (SEQ ID NO:4)
(v) the amino acid sequence Tau N-Alpha Acetyl-Met11-412 (SEQ ID NO:5);
(vi) the amino acid sequence Tau N-Alpha Acetyl-Met11-441 (SEQ ID NO:6);
(vii) the amino acid sequence Tau N-Alpha Acetyl-Met11-776 (SEQ ID NO:7); and
(viii) a fragment of at least 9 consecutive amino acids starting from the N-Alpha Acetyl methionine residue at position 11 of an amino acid sequence selected from amino acid sequences (i) to (vii).

4. The method according to claim 1 wherein the anti-Tau antibody does not bind to a non N-alpha-acetylated form of Methionine 11 Tau polypeptide (SEQ ID No 9) and/or a N-alpha-acetyl-Met1-Tau polypeptide (SEQ ID No 10).

5. (canceled)

6. The method according to claim 1, wherein the Tauopathy disorder is Alzheimer's disease.

7. An anti-Tau antibody wherein said antibody comprises:
(a) a heavy chain wherein the variable domain comprises:
a H-CDR1 having 90, 91, 92, 93, 94, 95, 96, 97, 98 or 99% of identity with the sequence set forth as SEQ ID NO: 11, and
a H-CDR2 having 90, 91, 92, 93, 94, 95, 96, 97, 98 or 99% of identity with the sequence set forth as SEQ ID NO: 12, and
a H-CDR3 having 90, 91, 92, 93, 94, 95, 96, 97, 98 or 99% of identity with the sequence set forth as SEQ ID NO: 13;
(b) a light chain wherein the variable domain comprises:
a L-CDR1 having 90, 91, 92, 93, 94, 95, 96, 97, 98 or 99% of identity with the sequence set forth as SEQ ID NO: 14, and
a L-CDR2 having 90, 91, 92, 93, 94, 95, 96, 97, 98 or 99% of identity with the sequence set forth as SEQ ID NO: 15, and
a L-CDR3 having 90, 91, 92, 93, 94, 95, 96, 97, 98 or 99% of identity with the sequence set forth as SEQ ID NO: 16
wherein the anti-Tau antibody binds to AcMet11-Tau polypeptide with substantially the same affinity as an antibody having a variable light chain domain (VL) and/or a variable heavy chain domain (VH) of the antibody 2H2D11.

8. The anti-Tau antibody according to claim 7 wherein said antibody comprises:
(a) a heavy chain wherein the variable domain comprises:
a H-CDR1 having a sequence set forth as SEQ ID NO: 11, and
a H-CDR2 having a sequence set forth as SEQ ID NO: 12, and
a H-CDR3 having a sequence set forth as SEQ ID NO: 13;
(b) a light chain wherein the variable domain comprises:
a L-CDR1 having a sequence set forth as SEQ ID NO: 14, and
a L-CDR2 having a sequence set forth as SEQ ID NO: 15, and
a L-CDR3 having a sequence set forth as SEQ ID NO: 16.

9. The anti-Tau antibody according to claim 8 wherein said antibody comprises:
a heavy chain wherein the variable domain has at least 70% or 80% or 90% of identity with the sequence set forth as SEQ ID NO:17 and
a light chain wherein the variable domain has at least 70% or 80% or 90% of identity with the sequence set forth as SEQ ID NO:18
wherein the antibody binds to AcMet11-Tau polypeptide with substantially the same affinity as an antibody having a variable light chain domain (VL) and/or a variable heavy chain domain (VH) of the antibody 2H2D11.

10. The anti-Tau antibody according to claim 9 wherein said antibody comprises:
a heavy chain wherein the variable domain has a sequence set forth as SEQ ID NO:17 and
a light chain wherein the variable domain has a sequence set forth as SEQ ID NO:18.

11. The anti-Tau antibody according to claim 7, wherein said anti-Tau antibody inhibits pathological seeding and/or aggregation of Tau protein.

12. (canceled)

13. (canceled)

14. (canceled)

15. (canceled)


SEQUENCE LISTING (20 sequences)

SEQ ID NO: 1 - protein, 342 aa, Homo sapiens [sequence omitted]

SEQ ID NO: 2 - protein, 371 aa, Homo sapiens [sequence omitted]

SEQ ID NO: 3 - protein, 373 aa, Homo sapiens
  MOD_RES (1)..(1): ACETYLATION
MEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 400 aa, Homo sapiens
  MOD_RES (1)..(1): ACETYLATION
MEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 402 aa, Homo sapiens
  MOD_RES (1)..(1): ACETYLATION
MEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 431 aa, Homo sapiens
  MOD_RES (1)..(1): ACETYLATION
MEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 766 aa, Homo sapiens
  MOD_RES (1)..(1): ACETYLATION
MEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 11 aa, Artificial
  Synthetic Tau N-Alpha Acetyl-Met11-21 (antigen)
  MOD_RES (1)..(1): ACETYLATION
MEDHAGTYGLG

SEQ ID NO: 9 - protein, 11 aa, Artificial
  Synthetic Tau Met11-21
MEDHAGTYGLG

SEQ ID NO: 10 - protein, 21 aa, Artificial
  Synthetic Tau N-Alpha Acetyl-Met1-21
  MOD_RES (1)..(1): ACETYLATION
MAEPRQEFEVMEDHAGTYGLG

SEQ ID NO: 11 - protein, 5 aa, Artificial
  Synthetic H-CDR1 of 2H2D11
DYYLS

SEQ ID NO: 12 - protein, 16 aa, Artificial
  Synthetic H-CDR2 of 2H2D11
NRADGYTTNYSASVKG

SEQ ID NO: 13 - protein, 9 aa, Artificial
  Synthetic H-CDR3 of 2H2D11
DNDHYAMDY

SEQ ID NO: 14 - protein, 16 aa, Artificial
  Synthetic L-CDR1 of 2H2D11
KSSQSLLYSNGKTYLN

SEQ ID NO: 15 - protein, 7 aa, Artificial
  Synthetic L-CDR2 of 2H2D11
QVSKLDP

SEQ ID NO: 16 - protein, 9 aa, Artificial
  Synthetic L-CDR3 of 2H2D11
LQGTYYPYT

SEQ ID NO: 17 - protein, 120 aa, Artificial
  Synthetic VH- of 2H2D11
EVRLVESGGGLVQPGGSLRLSCATSGFTFTDYYLSWVRQPPGKAFEWLGFIRNRADGYTTNYSASVKGRFTISRDNSQSILYLQMNTLRVEDSATYYCARDNDHYAMDYWGQGTSVTVSS

SEQ ID NO: 18 - protein, 112 aa, Artificial
  Synthetic VL- of 2H2D11
DVVMTQTPLSLSVTIGQPASISCKSSQSLLYSNGKTYLNWLQQRPGQAPKHLMYQVSKLDPGIPDRFSGSGSETDFILKISRVEAEDLGIYYCLQGTYYPYTFGGGTKLEIK

SEQ ID NO: 19 - DNA, 360 bp, Artificial
  Synthetic VH of 2H2D11 (nucleic acid sequence)
gaggtgaggctggtggagtctggaggaggcttggtacagcctgggggttctctgagactctcctgtgcaacttctgggttcaccttcactgattactacctgagctgggtccgccagcctccaggaaaggcatttgagtggttgggttttattagaaacagagctgatggttacacaacaaactacagtgcatctgtgaagggtcggttcaccatctccagagataattcccaaagcatcctctatcttcaaatgaacaccctgagagttgaggacagtgccacttattactgtgcaagagacaatgatcactatgctatggactactggggtcaaggaacctcagtcaccgtctcttca

SEQ ID NO: 20 - DNA, 336 bp, Artificial
  Synthetic VL of 2H2D11  (nucleic acid sequence)
gatgttgtgatgacccagactccactgtctttgtcggttaccattggacaaccagcctctatctcttgcaagtcgagtcagagcctcttatatagtaatggaaagacatatttgaattggttacaacagaggcctggccaggctccaaagcacctaatgtatcaggtgtccaaactggaccctggcatccctgacaggttcagtggcagtggatcagaaacagattttatacttaaaattagcagagtggaggctgaagatttgggaatttattactgcttgcaaggtacatattatccgtacacgttcggaggggggaccaagttggaaataaaa